SEQUENCE LISTING (22 sequences)

SEQ ID NO: 1 - DNA, 21 bp, Artificial Sequence
  siRNA targeting PKCdelta
ctttgaccaggagttcctgaa

SEQ ID NO: 2 - DNA, 20 bp, Artificial Sequence
  PCR primer
agtccaacatcaccatgcag

SEQ ID NO: 3 - DNA, 22 bp, Artificial Sequence
  PCR primer
ttccctttcctcgaactgattt

SEQ ID NO: 4 - DNA, 21 bp, Artificial Sequence
  PCR primer
gcaacaattcctggcgatacc

SEQ ID NO: 5 - DNA, 18 bp, Artificial Sequence
  PCR primer
ctccacggctaaccactg

SEQ ID NO: 6 - DNA, 22 bp, Artificial Sequence
  PCR primer
caagtatgagaagcctgggtct

SEQ ID NO: 7 - DNA, 20 bp, Artificial Sequence
  PCR primer
tgaagattggggtgtggaag

SEQ ID NO: 8 - DNA, 18 bp, Artificial Sequence
  PCR primer
aacgggaggtctgcaggg

SEQ ID NO: 9 - DNA, 20 bp, Artificial Sequence
  PCR primer
tgcttgtccttagtcctggc

SEQ ID NO: 10 - DNA, 22 bp, Artificial Sequence
  PCR primer
tgctcaacatctcccccttctc

SEQ ID NO: 11 - DNA, 22 bp, Artificial Sequence
  PCR primer
accaaatcccatatcctcgtcc

SEQ ID NO: 12 - DNA, 20 bp, Artificial Sequence
  PCR primer
gtaacccgttgaaccccatt

SEQ ID NO: 13 - DNA, 20 bp, Artificial Sequence
  PCR primer
ccatccaatcggtagtagcg

SEQ ID NO: 14 - DNA, 20 bp, Artificial Sequence
  PCR primer
gcaccgtcaaggctgagaac

SEQ ID NO: 15 - DNA, 20 bp, Artificial Sequence
  PCR primer
gccttctccatggtggtgaa

SEQ ID NO: 16 - RNA, 23 nt, Artificial Sequence
  fragment of PKCdelta 3′ UTR
gacuguggugacuucugcugcug

SEQ ID NO: 17 - RNA, 22 nt, Homo sapien
guguuugguaauacacgacgau

SEQ ID NO: 18 - RNA, 22 nt, Homo sapien
acauuugguacuacacgacgau

SEQ ID NO: 19 - RNA, 22 nt, Homo sapien
gcgguuauaaaugcacgacgau

SEQ ID NO: 20 - RNA, 21 nt, Homo sapien
cgguuauaaagacacgacgau

SEQ ID NO: 21 - RNA, 22 nt, Homo sapien
aaguuuuguacuuaacgacgac

SEQ ID NO: 22 - RNA, 21 nt, Homo sapien
uguuuggugucacacgacgac

(19) United States
(12) Patent Application Publication
(10) Pub. No.: US 2018/0066327 A1
(43) Pub. Date: Mar. 8, 2018

(54) Methods to Accelerate Wound Healing in Diabetic Subjects

(71) Applicant: Joslin Diabetes Center, Inc., Boston, MA (US)

(72) Inventors: George Liang King, Dover, MA (US); Mogher Khamaisi, Brookline, MA (US); Hillary A. Keenan, Welleley, MA (US)

(21) Appl. No.: 15/557,740

(22) PCT Filed: Mar. 14, 2016
(86) PCT No.: PCT/US16/22308

§ 371 Date: Sep. 12, 2017

Related U.S. Application Data
(60) Provisional application No. 62/133,222, filed on Mar. 13, 2015.
(60) Provisional application No. 62/233,289, filed on Sep. 25, 2015.

(51) Int. Cl. A61K 35/33; A61K 35/36; A61K 35/545; A61K 38/10; C12N 15/113; C12N 5/077; C12N 15/52

(52) U.S. Cl. CPC C12Y 207/10002; A61K 35/33; A61K 35/36; A61K 35/545; A61K 38/10; A61K 48/00; C12N 5/0656; C12N 15/52; C12N 2310/14; C12N 2310/141; C12N 2501/727; C12N 15/1137

(57) ABSTRACT

Methods of accelerating wound healing in diabetic subjects using autologous cell grafts treated to specifically inhibit Protein Kinase C delta (PKC6), as well as cells and compositions for use in these methods. Provided herein are methods for preparing cells for application to a wound in a diabetic subject. The methods include incubating the cells in the presence of an effective amount of a PKC6 inhibitor.

### FEDERALLY SPONSORED RESEARCH OR DEVELOPMENT

This invention was made with Government support under Grant Nos. 1R24DK090961-01 and DP3 DK094333-01 awarded by the National Institute of Diabetes and Digestive and Kidney Diseases (NIDDK) of the National Institutes of Health. The Government has certain rights in the invention.

### TECHNICAL FIELD

Methods of accelerating wound healing in diabetic subjects using autologous cell grafts treated to specifically inhibit Protein Kinase C delta (PKCδ), as well as cells and compositions for use in these methods.

### BACKGROUND

Poor wound healing of diabetic foot ulcers (DFU) are one of the most common and serious complications of diabetes leading to >80,000 amputations per year and acquiring high financial cost (Boulton A J, Lancet Volume 366, No. 9498, p 1719-1724, 2005; Brem and Tonic-Camic, J Clin Invest. 2007 May 1; 117(5): 1219-1222). Peripheral vascular disease, neuropathy, trauma, and reduced resistance to infection are recognized risk factors leading to the development of DFU, and poor wound healing (Brem and Tonic-Camic, J Clin Invest. 2007 May 1; 117(5): 1219-1222). Wound healing is a result of complex biological and molecular events of angiogenesis, cell adhesion, migration, proliferation, differentiation, and extracellular matrix (ECM) deposition (Michalik and Wahli, J Clin Invest. 2006;116(3):598-606). Abnormalities in all these steps have been reported in diabetes. However, identification of the mechanisms that contribute to poor wound healing in diabetes and characterization of the mechanisms as a therapeutic target have not been clarified.

Systemic changes characteristic of diabetes progression have been associated with increased risk of diabetic foot ulcer (DFU), including hyperglycemia (Bishop and Mudge, International wound journal. 2012; 9(6):665-76; Markuson et al., Advances in skin & wound care. 2009; 22(8):365-72), insulin resistance (Otranto et al., Wound repair and regeneration: official publication of the Wound Healing Society [and] the European Tissue Repair Society. 2013; 21(3):464-72), obesity (Seitz et al., Experimental diabetes research. 2010; 2010(476969; Pence et al., Advances in wound care. 2014; 3(1):71-9), and subsequent microvascular (Cheng et al., PloS one. 2013; 8(9):e75877; Walmsley et al., Diabetologia. 1989; 32(10):736-9; Ghanassia et al., Diabetes care. 2008; 31(7):1288-92; Zubair et al., Diabetes & metabolic syndrome. 2011; 5(3):120-5.) or macrovascular complications (McEwen et al., Journal of diabetes and its complications. 2013; 27(6):588-92), as well as localized factors. Multiple treatment modalities using cytokine replacement (Tsang et al., Diabetes care. 2003; 26(6):1856-61) and transplantation of keratinocytes or fibroblasts are effective in non-diabetic populations (Greer et al., Annals of internal medicine. 2013; 159(8):532-42; Hassan et al., Wound repair and regeneration: official publication of the Wound Healing Society [and] the European Tissue Repair Society. 2014; 22(3):313-25; Marston et al., Diabetes care. 2003; 26(6):1701-5), but their efficacy in patients with diabetes is diminished due to undetermined mechanisms (Greer et al., Annals of internal medicine. 2013; 159(8):532-42).

### SUMMARY

Provided herein are methods for preparing cells for application to a wound in a diabetic subject. The methods include incubating the cells in the presence of an effective amount of a PKCδ inhibitor.

Also provided herein are methods of treating a wound in a diabetic subject, e.g., for enhancing wound healing. The methods including providing a cell derived from the subject; incubating the cells in the presence of an effective amount of a PKCδ inhibitor; and administering the cells to the wound.

In some embodiments, the cells are keratinocytes, fibroblasts, or a combination thereof. In some embodiments, the cells are, or are derived from epithelial stem cells; human embryonic stem cells; induced pluripotent stem cells (iPS); bone-marrow-derived mesenchymal stem cells (BM-MSCs) or adipose-tissue-derived MSCs (ASCs).

In some embodiments, the cells are part of a split-thickness graft.

In some embodiments, the PKCδ inhibitor is selected from the group consisting of Rottlerin; PKC-412; and UCN-02; KAI-980, bisindolylmaleimide I, bisindolylmaleimide II, bisindolylmaleimide III, bisindolylmaleimide IV, calphostin C, chelerythrine chloride, ellagic Acid, Go 7874, Go 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, and D-erythro-sphingosine. In some embodiments, the PKCδ inhibitor is a dominant negative form of PKCδ.

In some embodiments, the PKCδ inhibitor is an inhibitory nucleic acid that specifically targets PKCδ or an oligonucleotide mimic that mimics a PKCδ miRNA selected from the group consisting of miR-15a, 15b, 16, 195, 424, and/or 497. In some embodiments, the inhibitory nucleic acid is 5 to 40 bases in length (optionally 12-30, 12-28, or 12-25 bases in length). In some embodiments, the inhibitory nucleic acid or oligonucleotide mimic is 10 to 50 bases in length. In some embodiments, the inhibitory nucleic acid comprises a base sequence at least 90% complementary to at least 10 bases of the PKCδ RNA sequence. In some embodiments, the inhibitory nucleic acid comprises a sequence of bases at least 80% or 90% complementary to, e.g., at least 5-30, 10-30, 15-30, 20-30, 25-30 or 5-40, 10-40, 15-40, 20-40, 25-40, or 30-40 bases of the RNA sequence. In some embodiments, the inhibitory nucleic acid comprises a sequence of bases with up to 3 mismatches (e.g., up to 1, or up to 2 mismatches) in complementary base pairing over 10, 15, 20, 25 or 30 bases of the RNA sequence. In some embodiments, the inhibitory nucleic acid comprises a sequence of bases at least 80% complementary to at least 10 bases of the RNA sequence. In some embodiments, the inhibitory nucleic acid comprises a sequence of bases with up to 3 mismatches over 15 bases of the RNA sequence. In some embodiments, the inhibitory nucleic acid is single stranded. In some embodiments, the inhibitory nucleic acid is double stranded.

In some embodiments, the inhibitory nucleic acid or oligonucleotide mimic comprises one or more modifications, e.g., comprising: a modified sugar moiety, a modified internucleoside linkage, a modified nucleotide and/or combinations thereof. In some embodiments, the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof. In some embodiments, the modified sugar moiety comprises a 2′-O-methoxyethyl modified sugar moiety, a 2′-methoxy modified sugar moiety, a 2′-O-alkyl modified sugar moiety, or a bicyclic sugar moiety. In some embodiments, the inhibitory nucleic acid comprises one or more of: 2′-OMe, 2′-F, LNA, PNA, FANA, ENA or morpholino modifications.

In some embodiments, the inhibitory nucleic acid is an antisense oligonucleotide, LNA molecule, PNA molecule, ribozyme or siRNA.

In some embodiments, the inhibitory nucleic acid is double stranded and comprises an overhang (optionally 2-6 bases in length) at one or both termini.

In some embodiments, the inhibitory nucleic acid is selected from the group consisting of antisense oligonucleotides, ribozymes, external guide sequence (EGS) oligonucleotides, siRNA compounds, micro RNAs (miRNAs); small, temporal RNAs (stRNA), and single- or double-stranded RNA interference (RNAi) compounds.

In some embodiments, the RNAi compound is selected from the group consisting of short interfering RNA (siRNA); or a short, hairpin RNA (shRNA); small RNA-induced gene activation (RNAa); and small activating RNAs (saRNAs).

In some embodiments, the antisense oligonucleotide is selected from the group consisting of antisense RNAs, antisense DNAs, and chimeric antisense oligonucleotides.

In some embodiments, incubating the cells in the presence of an effective amount of a PKCδ inhibitor comprises expressing a dominant negative PKCδ (dnPKCδ) in the cells.

In some embodiments, the methods include transfecting the cells with a viral vector encoding the dnPKCδ. In some embodiments, the viral vector is an adenoviral vector.

In some embodiments, the cells are administered in a carrier. In some embodiments, the carrier is, or is applied to, a membrane. In some embodiments, the carrier is liquid or semi-solid.

Also provided herein are isolated populations of cells prepared by a method described herein. As used herein, an “isolated” population of cells is a population of cells that is not in a living animal, e.g., a population of cells in culture or in a suspension. The cells may be purified, i.e., at least 40% pure, e.g., at least 50%, 60%, 70%, 80%, 90%, 95%, or 100% of a single type of cells, e.g., pure keratinocytes, fibroblasts, or a combination thereof, or cells derived from stem cells.

Further, provided herein are the isolated population of cells described or produced by a method described herein, for use in a method of treating a wound in a diabetic subject. In some embodiments, the cells were originally obtained from the subject to be treated (i.e., are autologous to the subject to be treated).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Effect of glucose, insulin, and hypoxia on VEGF expression. VEGF protein levels (A) and mRNA (B) in basal (cells incubated with DMEM medium only) or after incubation with100 nM insulin or after incubation for 16 hours in 5% O₂ hypoxic condition. VEGF protein levels secreted to the medium were measured using ELISA kit. This kit determines mainly VEGF₁₆₅. Real-time PCR using human VEGF primers detailed in table A were used to determine VEGF mRNA expression. Data presented as mean±SD obtained from 7 controls and 26 Medalists, each in triplicate. VEGF protein expression (C) after incubation of control and Medalist fibroblasts in 5.6 mM or 25 mM glucose for 24, 48, and 72 hours. Osmolality in 5.6 nM conditions was corrected using mannitol. Student's t-test or chi-square tests were used for two-way comparisons based on the distribution and number of observations of the variable. (D) VEGF protein levels in response to insulin in Medalists without CVD as compared to Medalists with CVD. (E) Hypoxia increased VEGF protein levels significantly in the Medalists without CVD compare to those with CVD.

FIGS. 2A-J. The effect of high glucose on fibroblast migration and ECM protein secretion. (A) A representative picture for scratch wound migration assay. (B) and (C) present the quantification after incubation with 25 mM glucose for 8 hours or 3 days, respectively. Osmolality in 5.6 nM conditions was corrected using mannitol. The images acquired for each sample analyzed quantitatively by using Image Pro-Plus software (Media Cybernetics). (D) Fibroblast migration determined in Matrigel invasion chamber. (E) Scratch wound migration assay in control and Medalist fibroblasts stimulated with 10 ng/ml PDGF-BB or 100 nM insulin for 12 h. Data presented as mean±SD obtained from 7 controls and 26 Medalists, each in triplicate. Representative immunoblots (F) and quantification for TGF-β (G) and fibronectin (H) protein levels in control and Medalist fibroblasts. (I) TGF-β and fibronectin (J) mRNA expression in Medalist fibroblasts. Basal mRNA expression in control fibroblasts was set to 1. Student's t-test or chi-square tests were used for two-way comparisons based on the distribution and number of observations of the variable.

FIGS. 3A-D. Medalist fibroblasts display impaired wound healing in vivo. (A) Macroscopically wound area surface not covered by an epithelial layer in wounds covered with Integra without human cells, Integra with control, or Integra with Medalist fibroblasts. (B) The percent of the open wound areas at day 9 and day 15 of the initial wound area. (C) H& E staining sections for open wound area and granulation tissues at day 15 post-initial wounding. D refers to dermis, and E refers to epidermis. Representative immunoblots for VEGF protein levels (D) and quantification (right panel) in the granulation tissues on day 15 post-wounding. VEGF mRNA levels. Data are mean±SD. n=12 for wounds treated with Integra without cells, n=7 for wounds treated with control fibroblasts, and n=12 for wounds treated with Medalist fibroblasts. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable. Scale bar: 50 um.

FIGS. 4A-C. Medalist fibroblasts display impaired wound healing in vivo. VEGF mRNA levels (A) in the granulation tissues on day 15 post-wounding. Extent of neovascularization in granulation tissues on day 15 post-wounding was assessed by CD31+ positive cells using immunohistochemistry (IHC) or immunofluorescence (IF) (B) and quantification (C). Data are mean±SD. n=12 for wounds treated with Integra without cells, n=7 for wounds treated with control fibroblasts, and n=12 for wounds treated with Medalist fibroblasts. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable. Scale bar: 50 um.

FIGS. 5A-H. Insulin signaling in control and Medalist fibroblasts. A representative immunoblot for p-AKT on s473 (A), p-AKT quantification (B), p-ERK (C) and p-ERK quantification (D) in control (C) or Medalist (M) fibroblasts, in basal state or after stimulation with 100 nM insulin or after stimulation with 10 ng/ml of BDGF-BB for 10 minutes. Phosphorylation on insulin receptor (Tyr 1135/1136) (upper panel in E), IRS-1 (Tyr 649 and 911) (middle panel in E), and AKT (s473) (lower panel in E) in the basal state and after stimulation with 100 nM insulin for 10 minutes in fibroblasts derived from controls or Medalists with or without CVD. Immunoblot quantifications are presented on the right side, where phosphorylation is on insulin receptor (F), IRS-1 (G), and AKT (H). Data are mean±SD, n=7 for the control fibroblast group, n=18 and 8 for the Medalist fibroblast group, with and without CVD, respectively. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable.

FIGS. 6A-G. Increased PKCδ expression and mRNA half-life in Medalists. A representative immunoblot for PKCδ (A), the protein quantification (B), and PKCδ mRNA, (C) in control and Medalist fibroblasts. Data are mean±SD, n=7 for the control fibroblast group and n=26 for the Medalists. PKC-α, -β1, and -β2 protein expression in the control (n=5) and Medalist (n=10) fibroblasts (D). (E) A representative immunoblot and the quantification (F) for PKCδ protein expression in control fibroblasts (N=7) and in fibroblasts of Medalists without CVD (N=8), and Medalists with CVD (N=18). The half-life for PKCδ mRNA (G) was determined by incubation of fibroblasts from control (n=7), and Medalists (n=10) with 5 ug/ml of actinomycin-D for 0 to 8 hours, followed by qRT-PCR analysis. Student's t-test or chi-square tests were used for two-way comparisons based on the distribution and number of observations of the variable.

FIGS. 7A-I. Knockdown of PKCδ improves insulin induced VEGF secretion. Adenoviral vector containing green fluorescent protein (Ad-GFP) or dominant negative PKCδ (Ad-dnPKCδ) infected Medalist fibroblasts under a fluorescent microscope (A). A representative immunoblot for PKCδ. (B) p-AKT after stimulation with insulin for 10 minutes (C), and VEGF protein levels after stimulation with 100 nM insulin for 16 hours (D) in Medalist fibroblasts infected with Ad-GFP or Ad-dnPKCδ. A representative immunoblot for PKCδ (E) and VEGF protein levels (F) in Medalist fibroblasts transfected with siRNA and stimulated with 100 nM insulin for 16 hours. A representative immunoblot for PKCδ (G) p-AKT after stimulation with 100 nM insulin for 10 minutes (H), and VEGF protein levels (I) after stimulation with 100 nM insulin for 16 hours in control fibroblasts infected with Ad-GFP or Ad-wtPKCδ. Data are mean±SD for n=10 in Medalist experiments and n=7 in the control experiments. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable.

FIGS. 8A-H. In vivo knockdown of PKCδ in Medalist fibroblasts improves wound healing, while increasing PKCδ expression in control fibroblasts delays wound healing after transplant in a non-diabetic host. Macroscopic wound area surfaces not covered by an epithelial layer (A), and H&E staining sections for open wound area and granulation tissues (B) at day 9 post-initial wounding in control cells infected with Ad-GFP or Ad-wtPKCδ. Macroscopic wound area surfaces not covered by an epithelial layer (C), and H&E staining sections for open wound area and granulation tissues (D) at day 9 post-initial wounding in fibroblasts derived from Medalists without CVD and infected with Ad-GFP or Ad-dnPKCδ. Macroscopical wound area surfaces not covered by an epithelial layer (E), and H&E staining sections for open wound area and granulation tissues (F) at day 9 post-initial wounding in fibroblasts derived from Medalists with CVD and infected with Ad-GFP or Ad-dnPKCδ. “D” and “E” in pictures B, D, and F refer to dermis epidermis, respectively. The percent of the open wound areas (G) and VEGF mRNA in granulation tissues (H) at day 9 after wounding in the different treatment groups is presented. Data are mean±SD, n=7 for the control fibroblast group, n=8 for Medalists with CVD, and n=8 for Medalists without CVD. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable. Scale bar: 50 um.

FIGS. 9A-D. In vivo knockdown of PKCδ in Medalist fibroblasts improves wound healing when transplanted in a diabetic host. Macroscopic wound area surfaces not covered by epithelial layer (A), and H&E staining sections for open wound area and granulation tissues at day 9 post-initial wounding (B) in control fibroblasts infected with Ad-GFP or Medalist fibroblasts infected with Ad-GFP or Ad-dnPKCδ. D and E in the pictures in (B) refer to dermis and epidermis, respectively. The percent of the open wound areas (C) and VEGF mRNA in granulation tissues (D) at day 9 after wounding in the different treatment groups is presented. Data are mean±SD, n=7 for the control fibroblast group, n=8 for the Medalist group. The criteria for selecting the cell lines for these experiments was completely random, and the selected subjects did not differ in any clinical or demographic characteristics from the rest of the patients. Student's t-test or chi-square tests were used for two-way comparisons based on the distribution and number of observations of the variable. Scale bar: 50 um.

FIGS. 10A-C. VEGF protein levels in Medalists with or without neuropathy (A), in patients with mild or severe kidney disease (B), and in patients with non-proliferative diabetic retinopathy (NPDR) or proliferative diabetic retinopathy (PDR) (C), in basal state or after incubation with100 nM insulin for 16 hours. VEGF protein levels secreted to the medium were measured using ELISA kit, each in triplicate. Data presented as mean±SD obtained from 7 controls and 12 without neuropathy and 12 with neuropathy, 13 with mild kidney disease (0 to 2A) and 11 with severe kidney disease (IIB to III), 13 with NPDR and 10 with PDR. The pathologic classifications for diabetic nephropathy used: Class I, glomerular basement membrane thickening: isolated glomerular basement membrane thickening and only mild, nonspecific changes by light microscopy that do not meet the criteria of classes II through IV. Class II, mesangial expansion, mild (IIA) or severe (IIB): glomeruli classified as mild or severe mesangial expansion but without nodular sclerosis or global glomerulosclerosis in more than 50% of glomeruli. Class III, nodular sclerosis at least one glomerulus with nodular increase in mesangial matrix without changes described in class IV.

FIG. 11. 12 hours starved confluent fibroblasts were stimulated with 10 ng/ml TGF for 24 hours. VEGF protein levels secreted to the medium were measured using ELISA kit. Data presented as mean±SD obtained from 6 controls and 6 Medalists, each in triplicate. Student's t-test or chi-square tests were used for two-way comparisons based on the distribution and number of observations of the variable.

FIGS. 12A-B. The nucleotide analogue bromodeoxy uridine (BrdU) incorporation into newly synthesized DNA stranded proliferating fibroblasts derived from controls and Medalists is presented in (A). Data are mean±SEM, n=7 for the control fibroblast group, n=26 for the Medalist group. (B) Following 24 hours fasting, fibroblasts derived from controls and Medalists were incubated with 10% FBS for an additional 24 hours. Cell-cycle distribution was analyzed by flow cytometry. Cells were fixed with 70% ethanol and stained with 50 μg/ml propidium iodide at 37° C. for 30 min. Stained cells (1×10) were quantified to determine the distribution of different cell cycle phases using Multicycle AV software (FACSAria, BD Biosciences, CA, USA). Data are mean±SD, n=7 for the control fibroblast group, n=10 for the Medalist group.

FIGS. 13A-C. H&E staining for Integra before transplanted. (A) Longitudinal section for Integra without fibroblasts. Longitudinal (B) and superficial (C) sections for Integra seeded with Medalist fibroblasts.

FIGS. 14A-I. Immune fluorescence (A-C) and immunohistochemistry (D-F) for human vimentin expression in mouse granulation tissue obtained from wounds that were not covered with Integra (A and D), in granulation tissue obtained from wounds transplanted with Integra without human fibroblasts (B and E), and in granulation tissue obtained from wounds transplanted with Integra seeded with human fibroblasts (C and F). Green represents human vimentin, and blue represents DAPI. Immunohistochemistry for MHC class 1 in mouse granulation tissue obtained from wounds that were not covered with Integra (G), in granulation tissue obtained from wounds transplanted with Integra without human fibroblasts (H), and in granulation tissue obtained from wounds transplanted with Integra seeded with human fibroblasts (I). n=5 for each treatment group.

FIGS. 15A-C. Representative immunoblots for PKCδ protein levels (A) and quantification (B) and PKCδ mRNA levels (C) in fibroblasts derived from skin biopsies obtained from four living type 1 diabetic patients (T1D) and four gender and age matched control healthy non-diabetic donors. Data are mean±SD. Student's t-test or chi-square tests were used for two-way comparisons based on the distribution and number of observations of the variable.

FIGS. 16A-C. Representative immunoblots for PKCδ protein levels (A) and quantification (B) and PKCδ mRNA levels (C) from living TID patients. The wound samples were obtained from discarded tissues from five active foot ulcers from type 1 diabetic patients and compared to tissues obtained from five gender and age matched non-diabetic patients who had surgery for other indications (eg: hammertoes, bunions and other complications). Data are mean±SD. Student's t-test or chi-square tests were used for two-way comparisons based on the distribution and number of observations of the variable.

FIGS. 17A-D. Representative immune-blots for PKCδ, α, and β2 isoforms in granulation tissue obtained 9 days after the initial wounding incision in STZ induced diabetic mice injected with STZ two weeks before wounding (A), and (B) the quantification of the blots. Representative immune-blot (C) and quantification (D) for tyrosine phosphorylation on PKCδ in granulation tissues obtained from control and STZ induced diabetic mice, after immunoprecipitation with anti-PKCδ antibody. Data are mean±SD, n=5 in each group.

FIGS. 18A-B. Representative immune-blots for p-Ser303 (A, lower panel) and p-Ser675 (A, upper panel) sites of IRS2 in fibroblasts derived from control, Medalist without or Medalist with CVD, and the quantifications corrected to total IRS 2 (B). Data are mean±SD, n=7 for the control fibroblast group, n=8 in each group of Medalists with or without CVD.

FIGS. 19A-D. VEGF levels in fibroblasts derived from controls or Medalists incubated with 100 nM insulin in the presence of 100 nM wortmanin (a PI3 kinase), or 10 μM PD98059 (a MAP kinase inhibitor (A), or with 100 nM RBX (a general PKCβ) (B), or with 10 mM GFX (a general PKC inhibitor) (C), or with 3 μM rottlerin (a PKCδ inhibitor) (D). Data are mean±SD, n=7 for the control fibroblast group, n=12 in the Medalist group.

FIG. 20. Extent of neovascularization in granulation tissues on day 15 post-wounding was assessed by CD31+ positive cells using immunofluorescence quantification. Data are mean±SD, n=7 for the control fibroblast group, n=8 for Medalists with CVD, and n=8 for Medalists without CVD. The criteria for selecting the cell lines for these experiments were completely random, and the selected subjects did not differ in any clinical or demographic characteristics from the rest of the patients. Student's t-test or chi-square tests were used for two-way comparisons based on the distribution and number of observations of the variable. Scale bar: 50 mm.

FIG. 21. Extent of neovascularization in granulation tissues on day 15 post-wounding was assessed by CD31+ positive cells using immunofluorescence quantification. Data are mean±SD, n=7 for the control fibroblast group, n=8 for the Medalist group. The criteria for selecting the cell lines for these experiments were completely random, and the selected subjects did not differ in any clinical or demographic characteristics from the rest of the patients. Student's t-test or chi-square tests were used for two-way comparisons based on the distribution and number of observations of the variable. Scale bar: 50 mm.

FIGS. 22A-B. miRNA expression was studied in the Medalists' fibroblasts compared to the controls using qPCR analysis. The non-coding RNA U6 was used for normalization of miRNA qPCR results. Data are mean±SD, n=5 for both the control and the Medalist groups. The criteria for selecting the cell lines for these experiments were completely random, and the selected subjects did not differ in any clinical or demographic characteristics from the rest of the patients. Student's t-test or chi-square tests were used for two-way comparisons based on the distribution and number of observations of the variable.

### DETAILED DESCRIPTION

Fibroblasts have emerged in recent years as a primary cell for regenerative therapy, due to their paracrine secretion of angiogenic factors, cytokines, and immunomodulatory substances (Darby et al., Clinical, cosmetic and investigational dermatology. 2014; 7:301-11; Driskell et al., Nature. 2013; 504(7479):277-81). However, similar to cytokine therapies, fibroblast therapy is clinically less effective in patients with diabetes than in non-diabetic persons (Thangapazham et al., International journal of molecular sciences. 2014; 15(5):8407-27), even with autologous transplant. These findings suggest the presence of metabolic memory in cultured fibroblasts from diabetic donors, or an ability of the diabetic milieu to rapidly induce cellular abnormalities in normal fibroblasts (Brandner et al., Diabetes care. 2008; 31(1):114-2; Brem et al., J Transl Med. 2008; 6:75).

Numerous factors are involved in the dynamic wound healing process. Platelet-derived growth factor (PDGF), tumor growth factor (TGF-β1, TGF-β2), vascular endothelial growth factor (VEGF), fibroblast growth factor (FGF), epidermal growth factor (EGF), tumor necrosis factor-alpha (TNF-α), and various inflammatory cytokines have crucial role in wound healing (Zgheib et al., Adv Wound Care (New Rochelle). 2014; 3(4):344-35). In addition, insulin action and hyperglycemia can affect key aspects of wound healing due to their role in cellular migration and proliferation.

The present study characterized the loss of insulin actions on wound healing in fibroblasts from diabetic subjects as insulin has been reported to affect the key steps in wound healing such as angiogenesis, and fibroblast migration and proliferation, (Maria H. M. Lima, PLOS one 2012; Xiao-Qi Wang, J Invest Dermatol. 2014). To identify potential mechanisms for diabetes induced impaired wound healing, the present study characterized the effect of hyperglycemia and the activation of protein kinase C (PKC) delta (PKCδ) on the function of fibroblasts of individuals with 50 or more years of type 1 diabetes (T1D) from the Joslin Medalist study (n=26) and age-matched controls (n=7) without diabetes. The extreme duration of diabetes in this group allowed us to determine clearly the impact of various vascular complications, mainly neuropathy, nephropathy, and retinopathy on fibroblast function and wound healing. In addition, these T1D patients are not obese (BMI<27), and without hyperinsulinemia or hyperlipidemia, which provide a unique opportunity to clarify the contribution of hyperglycemia, microvascular or macrovascular disease in the pathogenesis of impaired wound healing in diabetes.

In the present study, abnormalities were characterized in fibroblasts from a cohort of T1D patients, with well characterized complications. In addition, mechanisms were identified that may cause aberrations in fibroblast activity in wound healing. Without wishing to be bound by theory, presented herein is a potential therapy for correcting changes in insulin signaling that cause delays in wound healing in individuals with diabetes.

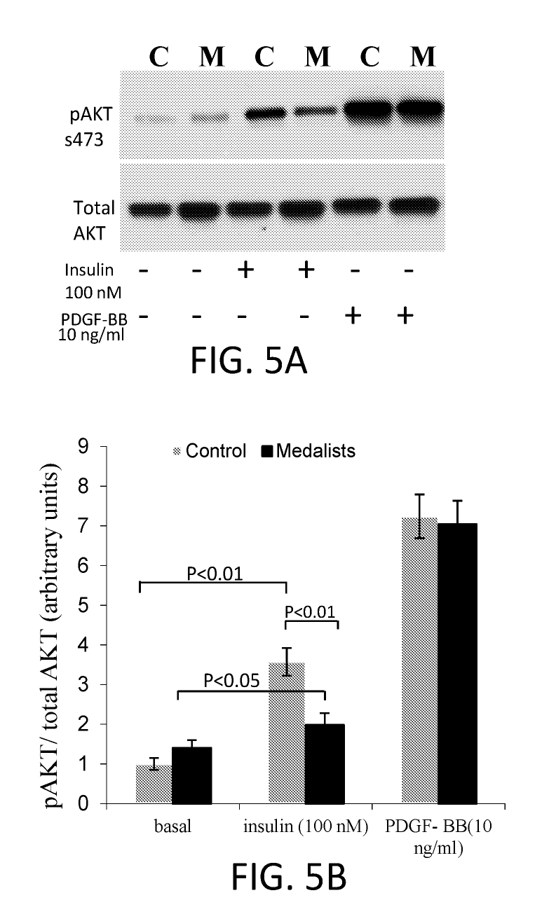

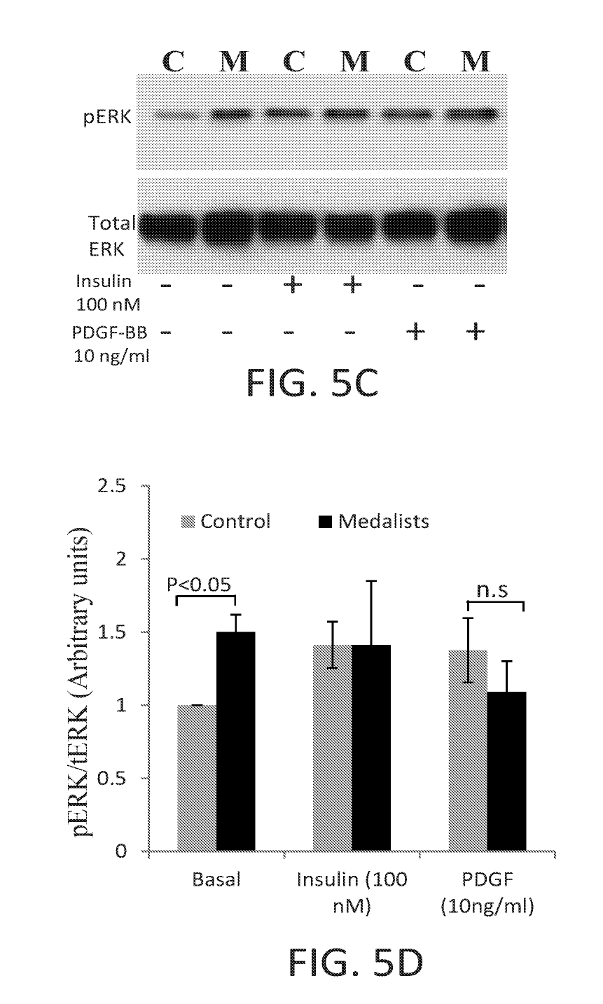

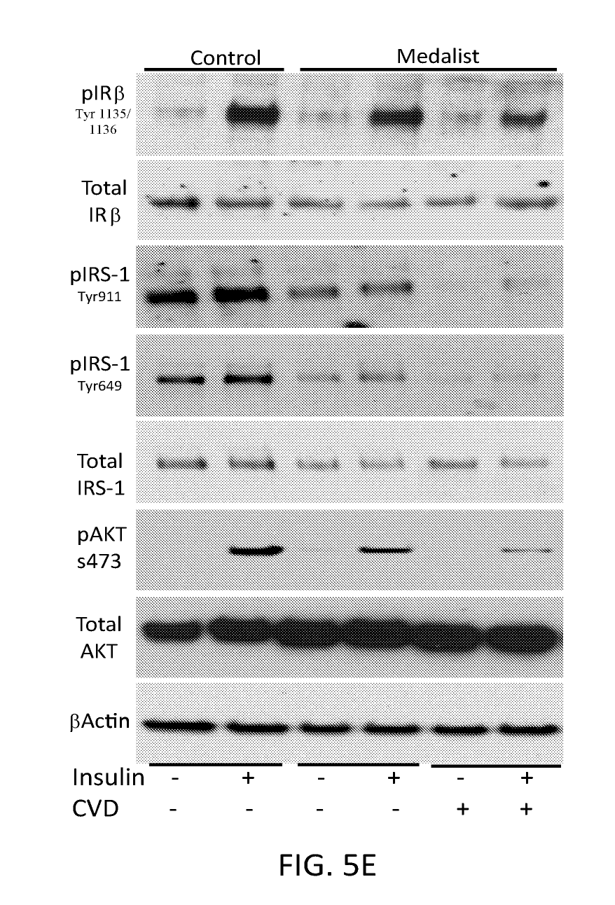

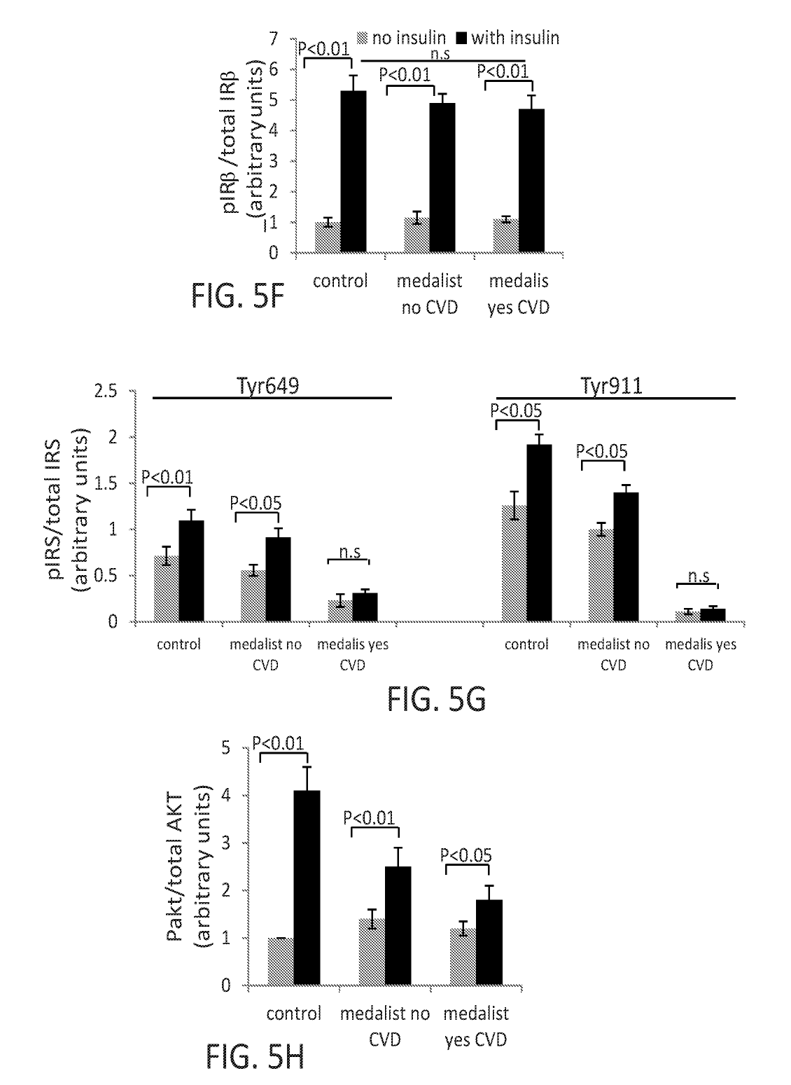

Fibroblasts were derived from individuals with diabetes for over 50 years (Joslin 50-Year Medalists). This enabled subgrouping of individuals according to protection from microvascular and cardiovascular complications after the plateau of microvascular diabetic complication incidence at approximately 30 years (Keenan et al., Diabetes. 2010; 59(11):2846-53; Sun et al., Diabetes care. 2011; 34(4):968-74). This is of great advantage since many of the neuropathic and vascular complications of diabetes are thought to confer independent risks in wound healing (Caanagh et 1., Lancet. 2005; 366(9498):1725-35; Veves et al., The Journal of clinical investigation. 2001; 107(10):1215-8). Thus, this unique cohort with extreme duration of disease and well-characterized micro- and macrovascular complications of diabetes enables analysis of the distinct contribution of each complication to fibroblast function and wound healing efficiency. The present experiments confirmed previous studies that fibroblasts derived from individuals with diabetes migrate less in response to various growth factors including PDGF and insulin (Lerman et al., The American journal of pathology. 2003; 162(1):303-12; Loots et al., Archives of dermatological research. 1999; 291(2-3):93-9; Loot et al., European journal of cell biology. 2002; 81(3):153-60). Interestingly, the fibroblasts from the Medalists did not exhibited resistance to PDGF-BB (FIG. 5B) indicating that the inhibition of insulin actions by PKCδ was limited to selective signaling pathways. In addition, the ability of Medalist fibroblasts to express VEGF in response to insulin and hypoxia was decreased, confirming previous reports (Lerman et al., The American journal of pathology. 2003; 162(1):303-12). Preliminary analysis suggested that abnormalities in the fibroblasts from the Medalists had some correlation to history of CVD and amputation. Abnormalities correlated with the presence of neuropathy, but not with the other microvascular complications: nephropathy or retinopathy. These findings suggest that wound healing may be induced by similar mechanisms as those that accelerate CVD in diabetic individuals. These in vitro abnormalities in diabetic fibroblasts were confirmed in vivo; fibroblasts from Medalists showed impaired function in wound healing in non-diabetic mice relative to control fibroblasts, demonstrated by decreases in VEGF expression and angiogenesis.

Abnormalities in wound healing in fibroblasts derived from the Medalists were related to decreased VEGF expression, especially in response to insulin and hypoxia. These findings suggest that the mechanisms for wound healing abnormalities associated with TID could be related to loss of insulin actions in fibroblasts. Metabolic changes such as hyperglycemia can inhibit insulin actions in several tissues in patients with T1D type 2 diabetes (Pang et al., J Clin Endocrinol Metab. 2013; 98(3):E418-28). This supports evidence in fibroblasts from other studies that demonstrated selective inhibition of insulin action in the IRS/PI3K/AKT cascade, without loss of activation of MAPK (Igarashi et al., The Journal of clinical investigation. 1999; 103(2):185-95). Thus, insulin's actions in the same cells can be preserved or inhibited selectively. Selective insulin resistance has been observed in many cardiovascular tissues including the myocardium (He et al., Arteriosclerosis, thrombosis, and vascular biology. 2006; 26(4):787-93), large arteries (He et al., Arteriosclerosis, thrombosis, and vascular biology. 2006; 26(4):787-93), endothelium (Rask-Madsen and King, Nature clinical practice Endocrinology & metabolism. 2007; 3(1):46-56), renal glomeruli, and other non-vascular tissues such as the liver (Vicent et al., The Journal of clinical investigation. 2003; 111(9):1373-80). The current study also demonstrated that selective insulin resistance appears to increase serine phosphorylation of IRS1/2. These are the same phosphorylation sites that we previously reported to be inhibitors of IRS 1/2 tyrosine phosphorylation, which interact with p85 of the PI3K pathway after insulin stimulation (Li et al., Circulation research. 2013; 113(4):418-27). The insulin stimulated tyrosine phosphorylation of the insulin receptor was similar between controls and Medalists, suggesting that selective insulin resistance is downstream to the receptors in the Medalist fibroblasts, as reported in vascular tissues (Li et al., Circulation research. 2013; 113(4):418-27; Shimomura et al., Molecular cell. 2000; 6(1):77-86). Without wishing to be bound by mechanism or theory, the present results demonstrate that PKCδ targets p-AKT and IRS1, thus inducing insulin resistance in the Medalist fibroblasts. Other signaling pathways regulated by p-AKT could also be involved, such as the forkhead boxO-1 (FOXO1) transcription factor, which has recently been found to be an important regulator of wound healing. In particular, FOXO1 has significant effects through regulation of transforming growth factor-β (TGF-β) expression and protecting keratinocytes from oxidative stress. In the absence of FOXO1, there is increased oxidative damage, reduced TGF-β1 expression, reduced migration and proliferation of keratinocytes and increased keratinocytes apoptosis leading to impaired re-epithelialization of wounds (Xu et al., Diabetes 2015; 64(1):243-56). As previously reported, hyperglycemia and angiotensin II, and possibly other causative factors such as oxidants and inflammatory cytokines, may play an important role in inducing selective insulin resistance, and in reducing the expression of VEGF and other cytokines (Maeno et al., The Journal of biological chemistry. 2012; 287(7):4518-30). The current findings suggest that selective insulin resistance in T1D is an important mechanism, that causes abnormality of fibroblast action in wound healing. This significantly extends previous reports that demonstrated the effect of loss of insulin action on the impairment of wound healing in diabetes. Goren et al. demonstrated that the expression of insulin signaling molecules is decreased in chronic wounds in diabetic ob/ob mice (Goren et al., The Journal of investigative dermatology. 2009; 129(3):752-64). This contrasts with our finding that the inhibition of insulin signaling is due to selective inhibition of signaling at the IRS1-PI3K step. Lima et al. also reported that the down-regulation of the IRS/PI3K/AKT pathway is important for wound healing (Lima et al., PloS one. 2012; 7(5):e36974). The finding that TGFβ action is inhibited by PKCδ activation suggests that other signaling pathways beside those involved with insulin could also be inhibited.

According to the current study, impaired signaling of insulin appears to be due to an increase in serine phosphorylation of the IRS proteins, which inhibits its tyrosine phosphorylation and actions on the PI3K/AKT pathway. As previously reported, the mechanism of the specific inhibition appears to be related to PKC activation (Park et al., Molecular and cellular biology. 2013; 33(16):3227-4). Here, the specific PKC isoform involved appears to be PKCδ rather than α and β, as we observed in endothelial cells (Li et al., Circulation research. 2013; 113(4):418-27; Maeno et al., The Journal of biological chemistry. 2012; 287(7):4518-30). Multiple factors have been shown to activate PKC in diabetes including hyperglycemia, elevation of free fatty acids, advanced glycation end products, oxidants, inflammation, and cytokines such as AngII (Geraldes and King, Circulation research. 2010; 106(8):1319-31; Rask-Madsen and King, Arteriosclerosis, thrombosis, and vascular biology. 2005; 25(3):487-96). However, the present finding is unusual in its demonstration of a persistent increased expression of PKCδ isoform even after culturing the fibroblasts for more than five passages in vitro, in fibroblast derived from biopsies obtained from living T1D, and from active wounds of living T1D, confirming the general applicability of this finding. We identified prolonged mRNA half-life as the mechanism for the increase in PKCδ expression, and as the stimulator of increased protein expression and activation. This contrasts with previous reports of other PKC isoforms in diabetes, which are activated by elevations in diacylglycerols (DAG) levels, resulting in activation rather than expression (Geraldes and King, Circulation research. 2010; 106(8):1319-31; Rask-Madsen and King, Arteriosclerosis, thrombosis, and vascular biology. 2005; 25(3):487-96). However, the primary molecular mechanism for the persistence increased PKCδ half-life is still unclear. Prolonged exposure to glucose, such as chronic hyperglycemia in the Medalists, may results in transcriptional de-regulation and changes in mRNA stability (Leibiger et al., Proc Natl Acad Sci USA. 1998; 95(16):9307-12), and contributes to the control of mRNA turnover. The predicted PKCδ miRNA regulators miR-15a, 15b, 16, 195, 424, and 497 were significantly decreased in the Medalists compared to the controls. No difference in miR-200a and miR-1227 were detected in Medalists compare to control fibroblasts. This could partially explain the increased protein levels in the Medalists compared to the controls. Future detailed studies are required to confirm which specific miRNAs can regulate PKCδ mRNA expression in fibroblasts and in-vivo in models of wound healing.

The finding that PKC activation plays an important role in the pathogenesis of impaired wound healing in diabetes is demonstrated by a series of studies that used either deletion or increased expression of PKCδ both in vitro and in vivo. Inhibition of PKCδ by knockdown or by small molecule inhibitors improved the fibroblast response to insulin and restored VEGF expression. On the other hand, increasing PKCδ expression in normal fibroblasts appears to mimic the abnormalities exhibited in fibroblasts derived from diabetic patients. The findings in vivo are very exciting since they show that normal fibroblasts, exogenously treated with PKCδ overexpression, inhibit wound healing. In contrast, fibroblasts derived from diabetic patients, exogenously treated with either inhibitors of PKCδ or with knockdown, improved wound healing. Further, the changes in PKCδ also correlated with the severity of abnormality in wound healing.

Inhibition or deletion of PKCδ ex vivo not only improved fibroblast function and wound healing in animals without diabetes, but also significantly improved the function of fibroblasts derived from diabetic patients, even when transplanted in a rodent model of diabetes due to severe insulin deficiency. This is surprising since it showed that exogenous modification of fibroblasts derived from diabetic patients can improve granulation tissue formation, angiogenesis, and wound healing. These findings suggest that activation of PKCδ is one means by which hyperglycemia and diabetes cause selective insulin resistance, and inhibit fibroblast actions for stimulating angiogenesis and granulation tissue formation. Therefore, exogenous treatment of PKCδ inhibition could be therapeutically effective in a diabetic state, despite the presence of hyperglycemia and other abnormalities such as oxidative stress and insulin resistance (Ruderman et al., The Journal of clinical investigation. 2013; 123(7):2764-72).

The capability of normalizing fibroblasts from diabetic hosts presents autogenic transplants of fibroblasts as a feasible and viable therapeutic method. The present experiments focused on the role of selective insulin resistance and assumed its normalization to be important in wound healing. However, our findings also suggest that the abnormalities of the response of fibroblasts to hypoxia could be a contributing factor. Previous studies identified activation of hypoxia inducible factor-1 alpha and its inhibition by p300 as an important pathway that is abnormal in fibroblasts from diabetic patients; the normalization of which could improve wound healing (Thangarajah et al., Proceedings of the National Academy of Sciences of the United States of America. 2009; 106(32):13505-10; Duscher et al., Proceedings of the National Academy of Sciences of the United States of America. 2015; 112(1):94-9). Thus, it is likely that abnormalities in wound healing, especially in fibroblasts, are caused by several important pathways that may be related to such phenomena as hyperglycemia, insulin resistance, and oxidative stress. However, a molecular mechanism is herein identified, namely the persistent manner of activation of PKCδ in the fibroblasts of diabetic patients, which leads to the selective inhibition of insulin action on the IRS/PI3K/AKT pathway. Persistent selective insulin resistance in fibroblasts leads to abnormal fibroblast functions, including expression of VEGF and migration of fibroblasts. This impairs wound healing that may result from abnormal fibroblasts or be induced by diabetes itself. However, the finding that all these aberrations can be normalized with exogenous PKCδ isoform inhibition in a diabetic in vivo model identifies a new therapeutic modality for treating diabetic patients using autologous transplant of modified fibroblasts.

Autologous Cells and Methods of Administration

Autogenic and allogenic transplants using fibroblasts is the mainstay treatment of chronic non-healing wounds. This is due to the multiple key roles of fibroblasts in wound healing, such as the production of growth factors and ECM protein, as well as the promotion of angiogenesis (Werner and Grose, Physiological reviews. 2003; 83(3):835-70; Bainbridge, Journal of wound care. 2013; 22(8):407-8, 10-12; Xuan et al., PloS one. 2014; 9(9):e108182; Hart et al., The international journal of biochemistry & cell biology. 2002; 34(12):1557-70; Weiss, Facial plastic surgery clinics of North America. 2013; 21(2):299-304). However, in diabetic states, there is evidence that autogenic and allogenic transplants involving fibroblasts are less efficacious than in non-diabetic individuals (Greer et al., Annals of internal medicine. 2013; 159(8):532-42). The present invention provides methods for accelerating wound healing in subjects, e.g., diabetic subjects, using cultured epithelial autografts (“CEAs”). In these methods, autologous cells (i.e., the subject's own cells) are treated to reduce expression or activity of PKCδ, and grafted onto the wound site.

In some embodiments, the cells are obtained by removing small skin samples, e.g., split thickness skin samples, are harvested from a site on the subject's body surface that is wound free, and epithelial cells are isolated from the sample. The epithelial cells (preferably keratinocytes) are then grown in culture and optionally expanded to a desired number of cells. Methods for isolating the cells and culturing them are well known in the art; see, e.g., Atiyeh and Costagliola, Burns. 2007; 33:405-413; Rheinwald and Green, Cell. 1975; 6:331-343; Green et al., Proc Natl Acad Sci USA. 1979; 76:5665-5668; Boyce, Burns. 2001; 27:523-533; Jones et al., Br J Plast Surg. 2002; 55:185-193; Gerlach et al., Principles of Regenerative Medicine No. 76. Gerlach J. Elsevier, ed. Burlington, Mass.: Elsevier/Academic Press; 2008. Innovative regenerative medicine approaches to skin cell-based therapy for patients with burn injuries. pp. 1298-1321; Gallico et al., N Engl J Med. 1984; 311:448-451; Green, Sci Am. 1991; 265:96-102; and Kamel et al., 2013, 217(3):533-555.

Alternatively, the cells can be keratinocytes derived from epithelial stem cells (see, e.g., Blanpain et al., Cell. 2007; 128:445-458; Lavoie et al., 2011; 37:440-447; Mcheik et al., Ann Chir Plast Esthet. 2009; 54:528-532; Rochat et al., In Handbook of Stem Cells (Second Edition), 2013, Chapter 65—Regeneration of Epidermis from Adult Human Keratinocyte Stem Cells, Pages 767-780); human embryonic stem cells (see, e.g., Guenou et al., Lancet. 2009; 374:1745-1753) or from induced pluripotent stem cells (iPS) (see, e.g., Uitto, J Invest Dermatol. 2011; 131:812-814). In embodiments, the cells can be, or can be derived from, bone-marrow-derived mesenchymal stem cells (BM-MSCs) or adipose-tissue-derived MSCs (ASCs); see, e.g., Menendez-Menendez et al., J Stem Cell Res Ther 2014, 4:1; Zografou et al., Ann Plast Surg. 2013 August; 71(2):225-32; and Castilla et al., Ann Surg. 2012 October; 256(4):560-72.

In some embodiments, the cells are part of a split-thickness autologous skin graft (STSG) or a dermal graft, and the methods include implanting the graft along with a pharmaceutical composition for the slow-release of a PKCδ inhibitor as described herein. Methods for obtaining and implanting an STSG or dermal graft are known in the art, see, e.g., Lindford et al., Burns. 2012; 38:274-282; Andreassi et al., Clin Dermatol. 2005; 23:332-337.

The methods described herein include the application (also referred to as administration or grafting) of cells treated with a PKCδ inhibitor, as described herein, onto a wound.

In some embodiments, the cells are formulated with a pharmaceutically acceptable carrier. The carrier can be solid, e.g., a membrane; liquid, e.g., in a liquid suspension that sets on or after contact with the wound; or semi-solid, e.g., in a hydrogel or other gel matrix. In preferred embodiments, the cells are applied along with a membrane carrier comprising a physiologically acceptable cell-support matrix, optionally with the cells disposed within the membrane. For example, the Integra™ membrane (Integra LifeSciences Corporation) is a Collagen-GAG matrix made of a 3 dimensional porous matrix of cross-linked bovine tendon collagen and glycosaminoglycan, optionally with a semi-permeable silicone membrane. See, e.g., U.S. Pat. No. 4,947,840, which discloses a biodegradable polymeric material for treating wounds. US20020146446 describes a surgical-medical dressing which uses a sandwich of two extracellular matrices grown on a composite composed of gelatin-fibronectin-heparan sulfate. A gel-matrix-cells integrated system that can be used in the present methods is described in US20100255052. Semisolid or flowable hydrogels comprising collagen/glycosaminoglycan (GAG) material are also known in the art, see, e.g., US 20110262503. Biocompatible dermal substitutes are described in US20050107876. In some embodiments the membrane is bioabsorbable, e.g., as described in US20070027414. In addition, see US 20110171180, which describes a microfabricated basal lamina analog that recapitulates the native microenvironment found at the dermal-epidermal junction (DEJ).

In embodiments where the cells are in a liquid carrier, the cells can be applied by any suitable method including pouring, painting, brushing, or spraying; devices for applying the cells are known in the art, e.g., as described in US20140107621. US20110311497 describes methods and devices suitable for producing a transplantable cellular suspension of living tissue suitable for grafting to a patient

The amount of cells adequate to accomplish the desired results can be determined based on the size and extent (e.g., depth) of the wound to be treated.

In some embodiments, the methods described herein can include co-administration with other drugs or pharmaceuticals, e.g., compositions for promoting wound healing or angiongensis, e.g., antibiotics to prevent infection, or stromal cell-derived factor-1α (SDF-1α) (Castilla et al., Ann Surg. 2012 October; 256(4):560-72).

The methods can include treating or preparing the wound to receive the cells, e.g., by cleansing or debriding the wound. In some embodiments,

PKCδ Inhibitors

The PKCd protein is a member of the Protein Kinase C family. In humans and in, this kinase has been shown to be involved in B cell signaling and in the regulation of growth, apoptosis, and differentiation of a variety of cell types. Alternatively spliced transcript variants encoding the same protein have been observed. PKCd has been identified as a therapeutic target for several indications, see, e.g., Yonezawa et al., Recent Pat DNA Gene Seq. 3(2):96-101 (2009); Shen, Curr Drug Targets Cardiovasc Haematol Disord. 3(4):301-7 (2003). Exemplary PKCd sequences in humans include GenBank Acc. No. NM 006254.3 (nucleic acid, for variant 1, the longer variant; both variants encode the same protein); NP_006245.2 (protein); NM_212539.1 (nucleic acid, for variant 1, the shorter variant, which lacks an exon in the 5′ UTR as compared to variant 1); and NP 997704.1 (protein). Human genomic sequence can be found at NC_000003.11 (Genome Reference Consortium Human Build 37 (GRCh37), Primary Assembly). PKCd is also known as MAY1; dPKC; MGC49908; nPKC-delta; and PRKCD.

The methods described herein include treating the autologous cells to inhibit the expression or activity of PKCδ before implantation. In some embodiments, the methods include inhibiting the expression or activity of PKCδ by at least 50%, or by at least 60%, at least 70%, 75%, 80%, or more, as compared to normal levels in a cell in the absence of a PKCδ inhibitor.

A number of PKCδ inhibitors are known in the art and include small molecule inhibitors as well as inhibitory nucleic acids and miRNA mimics. For example, PKCδ inhibitors include Rottlerin; PKC-412; and UCN-02; KAI-980, bisindolylmaleimide I, bisindolylmaleimide II, bisindolylmaleimide III, bisindolylmaleimide IV, calphostin C, chelerythrine chloride, ellagic Acid, Go 7874, Go 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, and D-erythro-sphingosine. See, e.g., US2008/0153903. Other small molecule inhibitors of PKC are described in U.S. Pat. Nos. 5,141,957, 5,204,370, 5,216,014, 5,270,310, 5,292,737, 5,344,841, 5,360,818, 5,432,198, 5,380,746, and 5,489,608, (European Patent 0,434,057), all of which are hereby incorporated by reference in their entirety. These molecules belong to the following classes: N,N′-Bis-(sulfonamido)-2-amino-4-iminonaphthalen-1-ones; N,N′-Bis-(amido)-2-amino-4-iminonaphthalen-1-ones; vicinal-substituted carbocyclics; 1,3-dioxane derivatives; 1,4-Bis-(amino-hydroxyalkylamino)-anthraquinones; furo-coumarinsulfonamides; Bis-(hydroxyalkylamino)-anthraquinones; and N-aminoalkyl amides, 24143-Aminopropyl)-1H-indol-3-yl]-3-(1H-indol-3-yl)maleimide, 2-[1-[2-(1-Methylpyrrolidino)ethyl]-1H-indol-3-yl]-3-(1H-indol-3-yl)maleimide, Go 7874. Other known small molecule inhibitors of PKC are described in the following publications (Fabre, S., et al. 1993. Bioorg. Med. Chem. 1, 193, Toullec, D., et al. 1991. J. Biol. Chem. 266, 15771, Gschwendt, M., et al. 1996. FEBS Lett. 392, 77, Merritt, J. E., et al. 1997. Cell Signal 9, 53., Birchall, A. M., et al. 1994. J. Pharmacol. Exp. Ther. 268, 922. Wilkinson, S. E., et al. 1993. Biochem. J. 294, 335., Davis, P. D., et al. 1992. J. Med. Chem. 35, 994), and belong to the following classes: 2,3-bis(1H-Indol-3-yl)maleimide (Bisindolylmaleimide IV); 24143-Dimethylaminopropyl)-5-methoxyindol-3-yl]-3-(1H-indol-3-yl)maleimide (Go 6983); 2-{8-[(Dimethylamino)methyl]-6,7,8,9-tetrahydropyrido[1,2-a]indol-3-yl}-3-(1-methyl-1H-indol-3-yl)maleimide (Ro-32-0432); 2-[8-(Aminomethyl)-6,7,8,9-tetrahydropyrido[1,2-a]indol-3-yl]-3-(1-methyl-1H-indol-3-yl)maleimide (Ro-31-8425); and 3-[1-[3-(Amidinothio)propyl-1H-indol-3-yl]-3-(1-methyl-1H-indol-3-yl)maleimide Bisindolylmaleimide IX, Methanesulfonate (Ro-31-8220) all of which are also hereby incorporated by reference in their entirety.

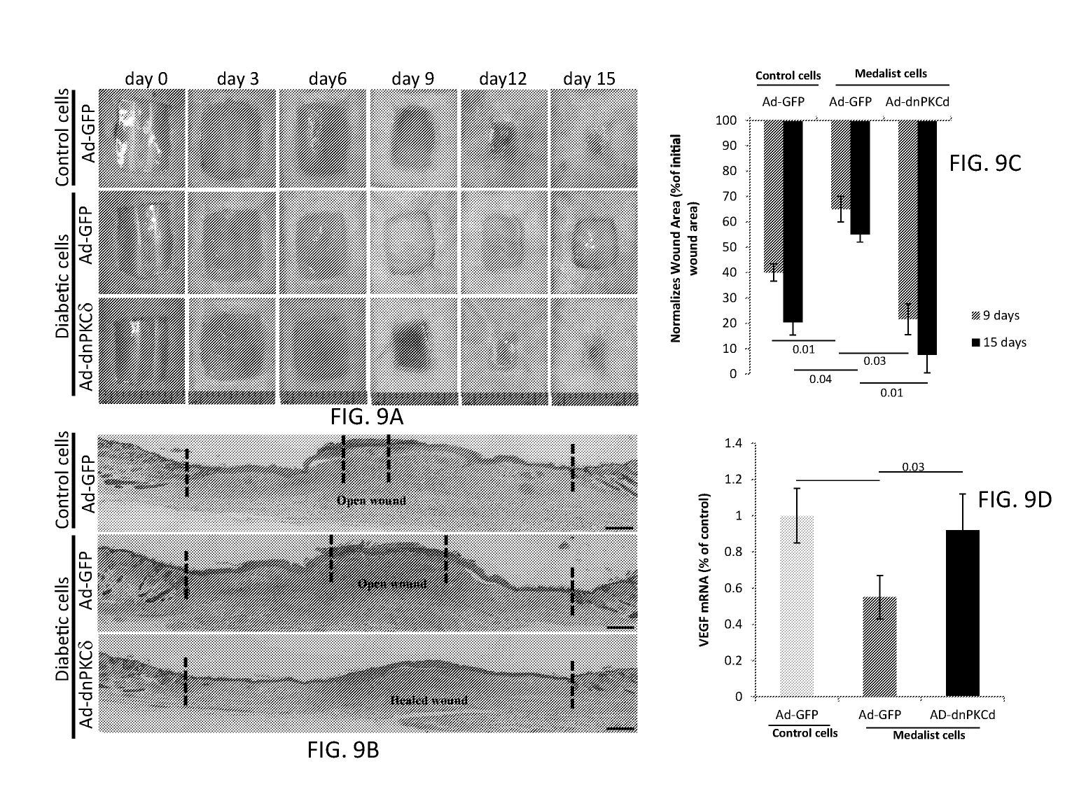

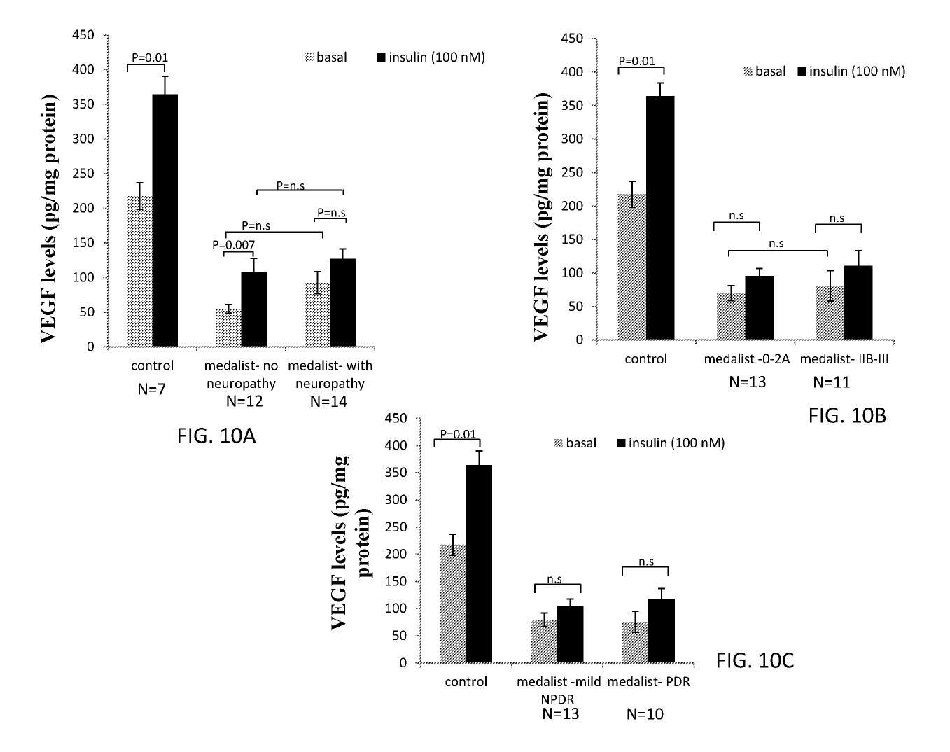

In some embodiments, the PKCδ inhibitor is a peptide inhibitor or peptidomimetic thereof, e.g., comprising 4 to 25 residues of the first variable region of PKCd. In some embodiments, the PKCδ inhibitor is KAI-9803 (KAI Pharmaceuticals, Inc., South San Francisco, Calif.); described in WO2009/029678, and other inhibitors listed therein, e.g., in Table 1 thereof. In some embodiments, the PKCδ inhibitor is KID1-1, amino acids 8-17 [SFNSYELGSL]) conjugated reversibly to the carrier peptide Tat (amino acids 43-58 [YGRKKKRRQRRR]) by disulfide bond as described in [9,11] (KAI Pharmaceuticals). Other peptide inhibitors are known in the art, e.g., as described in U.S. Pat. No. 6,855,693, U.S. Patent Application Publication Nos. 2004/204364, 2003/211109, 2005/0215483, and 2006/0153867; WO2006017578; and U.S. Provisional Patent Application Nos. 60/881,419 and 60/945,285. In some embodiments, the PKCδ selective inhibitor is Rottlerin (mallatoxin) or a functional derivative thereof. The structure of Rottlerin is shown in FIG. 9 of US2009/0220503. In some embodiments, the PKCδ selective inhibitor is Balanol or a Balanol analog (i.e., perhydroazepine-substitution analogs). Balanol is a natural product of the fungus Verticillium balanoides (Kulanthaivel et al., J Am Chem Soc 115: 6452-6453 (1993)), and has also been synthesized chemically (Nicolaou et al., J. Am. Chem Soc 116: 8402-8403 (1994)). The chemical structure of balanol is shown in FIG. 10 of US 2009/0220503. Balanol and perhydroazepine-substitution analogs are disclosed in US 2009/0220503 (see, e.g., Table 2 therein). Other derivatives based upon the structure of mallatoxin or balanol can be made, wherein the core structure is substituted by C.sub.1-C.sub.6 groups such as alkyl, aryl, alkenyl, alkoxy, heteroatoms such as S, N, O, and halogens. Additional PKCd-specific inhibitors are described in Int'l Pat. Appl. Nos. WO2004078118, WO2009029678, and U.S. Pat. Nos. 6,828,327, 6,723,830, 6,686,373 and 5,843,935. See also Hofmann, The FASEB Journal 11(8):649-669 (1997). A dominant-negative PKCδ (PKCδ-kinase dead (PKCδ-KD)), is also known in the art; see, e.g., Carpenter et al., The Journal of Biological Chemistry, 276:5368-5374 (2001).

In some embodiments, the inhibitor of PKCδ is an inhibitory nucleic acid that is complementary to PKCδ. Exemplary inhibitory nucleic acids for use in the methods described herein include antisense oligonucleotides and small interfering RNA, including but not limited to shRNA and siRNA. The sequence of PKCδ is known in the art; in humans, there are 2 isoforms:

| Variant | Nucleic Acid | Protein | Notes |
|---|---|---|---|
| Variant 1 | NM_006254.3 | NP_006245.2 | variant (1) represents the |
| | | | longer transcript. Both |
| | | | variants encode the same |
| | | | protein. |
| Variant 2 | NM_212539.1 | NP_997704.1 | variant (2) lacks an exon |
| | | | in the 5′ UTR compared to |
| | | | variant 1. Both variants |
| | | | encode the same protein. |

Alternatively or in addition, the inhibitor of PKCδ is a nucleic acid that mimics a PKCδ miRNA regulator, e.g., miR-15a, 15b, 16, 195, 424, and/or 497, and thereby decreases PKCδ expression. Exemplary sequences of these miRNAs are known in the art and shown in Table 2.

Inhibitory Nucleic Acids

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the PKCδ sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within an PKCδ sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect. Antisense molecules targeting PKCδ are described in U.S. Pat. No. 6,339,066; U.S. Pat. No. 6,235,723; and WO0070091.

siRNA/shRNA

In some embodiments, the nucleic acid sequence that is complementary to an PKCδ RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

siRNA targeting PKCδ has been described, see, e.g., Xia et al., Cell Signal. 2009 April; 21(4): 502-508 (CTTTGACCAGGAGTTCCTGAA, SEQ ID NO:1).

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261: 1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 mM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

miRNA Mimics

In some embodiments, the PKCδ inhibitor is a miRNA mimic, i.e., an oligonucleotide that has the same sequence as miRNA that regulates PKCδ. The mimics can also be modified, e.g., chemically modified. For example, a miRNA mimic for use in the methods described herein can include a nucleotide sequence identical to an miRNA sequence. Preferred miRNA sequences include PKCδ miRNA regulators miR-15a, 15b, 16, 195, 424, and 497. Exemplary sequences are shown in Table 2.

Modified Nucleic Acids

In some embodiments, the nucleic acids (both mimics and inhibitory nucleic acids) used in the methods described herein are modified, e.g., comprise one or more modified bonds or bases. A number of modified bases include phosphorothioate, methylphosphonate, peptide nucleic acids, or locked nucleic acid (LNA) molecules. Some nucleic acids are fully modified, while others are chimeric and contain two or more chemically distinct regions, each made up of at least one nucleotide. These nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

The nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the activity, e.g., the inhibitory activity, of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences described herein can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### EXAMPLES

The invention is further described in the following examples, which do not limit the scope of the invention described in the claims.

Material and Methods

The following materials and methods were used in the examples set forth herein.

Antibodies and Other Reagent

Antibodies used for western immuno-blotting included anti-β-actin (sc-1616), PKCα (H-7) (sc-8393), PKCβ1 (C-16) (sc-209), PKCβ2 (C-18) (sc-210), Fibronectin (A-11) (sc-271098), TGFβ (3C11) (sc-130348), VEGF (A-20) (sc-152), pIRS-1 (tyr632) (sc-17196), Insulin Receptor beta (IRβ) (sc-711), goat anti-mouse (sc-2031) and anti-rabbit IgG (sc-2004), all were purchased from Santa Cruz Biotechnology Inc (Santa Cruz, Calif.). Anti PKCδ (#2058s), p-Insulin Receptor beta (#3025s), IRS-1 (#2390s), rabbit polyclonal antibodies for phosphorylated and total AKT and ERK obtained from Cell Signaling (Danvers, Mass.). Antibodies for IRS-1 p-Tyr (911) and p-Tyr (649) purchased from Sigma (St. Louis, Mo.). Anti-Vimentin/LN6 Ab was obtained from Calbiochem (San Diego, Calif.). Anti-mouse CD-31 (DIA-310) was obtained from Dianova GmbH (Hamburg, Germany). Anti-MHC Class 1 (NB110-57201) was purchased from NOVUS (Littleton, Colo.). Anti-PDGF BB was purchased from abcam (Cambridge, Mass.).

Ruboxistaurin (RBX) was purchased from Millipore (Billerica, Mass.). 2[1-(3-dimethylaminopropyl)-1H-indol-3-yl]-3-(1H-indol-3-yl)-maleimide (GFX) was obtained from Calbiochem (La Jolla, Calif.). Rottlerin, PD098059 and, wortmannin were obtained from Sigma (St. Louis, Mo.). Plasmid transfections used Lipofectamine™ 2000 was purchased from Invitrogen by Life Technologies (Grand Island, N.Y.).

Dulbecco's Modified Eagle's Medium (DMEM) was provided by Joslin Media Core.

Fetal bovine serum (FBS), phosphate-buffered saline (PBS), and penicillin-streptomycin were obtained from Invitrogen (Grand Island, N.Y.). All other reagents employed including bovine serum albumin (BSA), 2,4,6-trinitrobenzenesulfonic acid, EDTA, heparin, leupeptin, phenylmethylsulfonyl fluoride, aprotinin, leupeptin, PDGF-BB, d-glucose, d-manitol, and Na₃VO₄ were purchased from Sigma-Aldrich, unless otherwise stated.

Human Studies in the Medalist Patients

Details of the Medalists Study have been described extensively elsewhere (Keenan et al., Diabetes. 2010 November; 59(11):2846-53; Sun et al., Diabetes Care. 2011 April; 34(4):968-74; Hernandez et al., Diabetes Care. 2014 August; 37(8):2193-201). Individuals who had documented 50 or more years of insulin use for type 1 diabetes were invited to participate in a baseline visit. Informed consent was obtained from all subjects prior to participation in the study and the Joslin Diabetes Center Committee on Human Studies reviewed and approved the protocol of this study.

Assessment of Complication Status

Nephropathy, retinopathy and neuropathy status were defined previously (King G L, 2009, 2011, and 2014). Briefly, diabetic nephropathy (DN) was defined by an estimated glomerular filtration rate (eGFR) of <45 mL/min/1.73 m2. A dilated eye examination was performed and retinopathy status was graded using guidelines from the Early Treatment Diabetic Retinopathy Study (ETDRS). Proliferative diabetic retinopathy (PDR) was defined as an ETDRS≧60 (Ophthalmology 1991, 823-833). The Michigan Neuropathy Screening Instrument was used to assess neuropathy; scores>2 were considered positive (Feldman E L, DC 1994). Cardiovascular disease (CVD) status was based on self-reported history of coronary artery disease, angina, heart attack, prior cardiac or leg angioplasty, or bypass graft surgery. Coronary artery disease (CAD) consists of being told by a clinician that they have coronary artery disease, angina, heart attack, history of cardiac angioplasty or bypass graft surgery. Peripheral vascular disease (PVD) consists of self-reported history of peripheral vascular disease, leg angioplasty, or leg bypass graft surgery.

Post-Mortem Samples

At the time of clinical characterization, individuals were asked to donate their organs after death. Consent was sought for skin, kidneys, ocular globes, left ventricle, aorta, bone, and pancreas. At the time of imminent death study staff was notified of the participant's condition by a 24-hour phone line. The coordinating center was then notified and the organ procurement organization (The National Disease Research Interchange (NDRI)) was called. A skin sample, 2.0×2.0 to 5.0×5.0 cm, from the abdomen or forearm was placed in DMEM+antibiotics and shipped on wet ice.

Human Primary Fibroblast Derivation and Culture

Skin were obtained from 26 Medalists with various complications and from 7 age-matched non-diabetic controls during post-mortem period. Primary fibroblast cultures were derived from human skin samples, sustained in DMEM (10-027, Cellgro Inc.) supplemented with 10% heat inactivated fetal bovine serum, over a period of 4 weeks, in a 6-well plate, with media supplementation every other day. Subsequently, as fibroblasts emerged from the primary explant, a brief trypsinization (0.25% Trypsin) was used to separate and further expand the cells in a 10 cm² plate. In all experiments fibroblasts were used between passages 2-5.

In another set of experiments, fibroblasts were derived from biopsies obtained from four living T1D patients and four age and gender mathech control non-diabetic subjects.

Human Samples from Active Foot Ulcers

De-identified discarded skin specimens were collected from 50 to 65 year-old subjects who underwent elective foot surgery at the Foot Center and Vascular Surgery clinic at the Joslin/Beth Israel Deaconess Medical Center. Subjects were divided into 2 groups: i) control group (non-diabetic subjects who had elective surgery (eg: hammertoes, bunions and other foot surgeries); ii) diabetic foot ulcer group (diabetic patients with an active foot ulcer). They were matched for age and gender. The skin specimens were collected at the time of the surgery in the operating room. Only the specimens that were determined by the operating surgeon to be discarded specimens evaluation were collected and used for this study. All procedures were approved by the Beth Israel Deaconess Medical Center Institutional Review Board (IRB).

Wound Model in Mice

All of the animal experiments were performed in compliance with the Joslin Diabetes Center Statement for the Use of Animals in Diabetic Research. For in vivo wound healing experiments 8 week old male nude mice (nu/nu, 002019), were used from Jackson Laboratories (Bar Harbor, Me.).

On the day of the surgery (Day 0), mice were anesthetized and the dorsal skin was marked using a standardized 1.0 cm² square template. A full-thickness wound on the dorsal area was created by excising a 1 cm×1 cm square of skin (epidermis, dermis, and underlying panniculus carnosus).

For the transfer of human fibroblast cells into the animal wound, we used Integra bilayer matrix wound dressing as a dermal regeneration template, donated by Integra LifeSciences Corporation (Plainsboro, N.J.). This is a gelatin based scaffold produced by a cryogelation technique, with attached silicone pseudoepidermal layer for wound repair purposes. The scaffold possessed an interconnected macroporous structure with a pore size distribution of 131±17 μm at one surface decreasing to 30±8 μm at the attached silicone surface (Shevchenko et al., Acta Biomater. 2014 July; 10(7):3156-66).

Fibroblasts (10⁵ cells) originated from Medalists or control subjects were plated on 1.0×1.0 cm piece of Integra in six well plate a day before the surgery. After 16-20 h the fibroblast-seeded Integra membranes were transplanted on the animal wound. The Integra was sutured onto the wound, ensuring that its porous bottom surface was in contact with the wound bed. Once dry, the wound area was covered with semi occlusive transparent polyurethane dressing (Tegaderm™, 3M, St. Paul, Minn.). Three days post-surgery, the silicone outer layer of the Integra was removed. Each three days the Tagaderm was replaced.

The experimental groups included: (a) wound without Integra (n=12); (b) wound with Integra without cells (n=12); (c) wound with Integra with controls cell (n=12); and (d) wound with Integra with Medalist cell (n=12).

On days 0, 3, 6, 9, 12, and 15 post-surgery, the re-epithelialization of the wound was monitored macroscopically (based on the absence of redness and fluid exudate) and wounds open area were photographed digitally. In days 9 or 15 post-surgery, wounds from 6 animals in each group were harvested as previously described (Succar et al., Plast Reconstr Surg. 2014 September; 134(3):459-67).

In another series of experiments, fibroblasts from controls donors were transfected with either adenoviral vectors containing green fluorescent protein (GFP, Ad-GFP), or wild-type PKCδ isoforms (Ad-wtPKCδ). Medalists' fibroblasts were transfected with either Ad-GFP or dominant negative PKCδ isoforms (Ad-dnPKCδ, comprising a point mutation at K378R) (Geraldes et al. Nat Med. 2009 November; 15(11):1298-306; Kaneto et al, J. Biol. Chem. 277:3680-3685 (2002)).

The experimental groups included: (a) controls fibroblast transfected with Ad-GFP or; (b) with Ad-wtPKCδ; (c) fibroblasts from Medalists without CVD transfected with Ad-GFP or; (d) with Ad-dnPKCδ; (e) fibroblasts from Medalists with CVD transfected with Ad-GFP or; (f) with Ad-dnPKCδ. After 24-48 h transfection, the equal numbers of cells were plated on Integra and transplanted onto the nude mice as described earlier.

To detect the effect of diabetes on wound healing in vivo in mice, diabetes was induced in 8 week old nude mice by streptozotocin (STZ) as described previously (Mima et al., Invest Ophthalmol Vis Sci. 2012 Dec. 19; 53(13):8424-32). Two weeks after STZ injection, animals with glucose levels above 400 mg % were used. On day 0 wound was produced as described earlier. On day 9, animals were scarified and granulation tissue was collected and frozen in −80 C until used for protein and mRNA analysis (Heit et al., Plast Reconstr Surg. 2013 November; 132(5):767e-776e).

Tissue morphometric analysis—To assess the macroscopic wound area, digital macroscopic images were analyzed using NIH ImageJ software v1.40g (ImageJ, NIH, Bethesda, Md.). Standardized photographs were taken on the day of wounding and each three days during the follow-up. Reepithelialization and open wound raw surface were measured as a percentage of the initial wound area as published previously (Erba and Orgill, Ann Surg. 2011 February; 253(2):402-9).

Histological processing—Excised tissues were fixed and stored at 4° C. until final processing. Wound tissues were stained using the hematoxylin & eosin (H&E) protocol. The histological images were photographed using a Nikon Labophot (Melville, N.Y.) microscope equipped with a Polaroid DMC2 color camera (Concord, Mass.) using analysis software version v2.1. Images were taken in the center of each histological section at ×4, ×10, and ×40 magnifications.

Immunoblot Analyses

Fibroblasts with passage≦5 were grown and expanded in 10 cm plate with DMEM supplemented with 10% FBS. Cells were stimulated with the conditions and compounds as indicated after overnight starvation in DMEM with 0.1% BSA without FBS. Cells were lysed and protein amounts were measured with BCA kit (Bio-Rad, Hercules, Calif.). Protein lysates (20-30 μg) were separated by SDS-PAGE, transferred, blocked and detected as we described before (Park et al., Mol Cell Biol. 2013 August; 33(16):3227-41). The signal intensity was quantified using ImageJ software (SynGene, Frederick, Md.). For immune precipitation, tissue lysates were incubated with the appropriate antibodies followed by the addition of protein A/G Sepharose beads (Santa Cruz Biotechnology, Inc., Santa Cruz, Calif.). The precipitated proteins were subjected to SDS-PAGE followed by immunoblotting with the appropriate antibodies as described before (Park et al., Mol Cell Biol. 2013 August; 33(16):3227-41).

Real Time PCR Analysis

Real-time PCR was performed to evaluate mRNA expressions of VEGF, PDGF-B, Fibronectin, and PKCδ in cultured fibroblast and mice granulation tissue as we described before (Geraldes et al., Nat Med. 2009 November; 15(11):1298-306). PCR primers and probes are detailed in Table A. Human 36B4 or 18S ribosomal RNA expressions as indicated were used for normalization.

TABLE A

| | | SEQ ID | | SEQ ID |
|---|---|---|---|---|
| Gene | Forward (5′-3′) | NO: | Reverse (5′-3′) | NO: |
| Human VEGF | AGTCCAACATCACCATGCAG | 2 | TTCCCTTTCCTCGAACTGATTT | 3 |
| Human PDGF-BB | GCAACAATTCCTGGCGATACC | 4 | CTCCACGGCTAACCACTG | 5 |
| Human | CAAGTATGAGAAGCCTGGGTC | 6 | TGAAGATTGGGGTGTGGAAG | 7 |
| Fibronectin | T | | | |
| Human PKCδ | AACGGGAGGTCTGCAGGG | 8 | TGCTTGTCCTTAGTCCTGGC | 9 |
| Human 36B4 | TGCTCAACATCTCCCCCTTCTC | 10 | ACCAAATCCCATATCCTCGTCC | 11 |
| Human 18S | GTAACCCGTTGAACCCCATT | 12 | CCATCCAATCGGTAGTAGCG | 13 |
| ribosomal RNA | | | | |
| Human GABDH | GCACCGTCAAGGCTGAGAAC | 14 | GCCTTCTCCATGGTGGTGAA | 15 |

Half-life study of mRNA—Cells were treated with 5 mg/ml Actinomycin-D for the indicated times. Total RNA isolation, cDNA synthesis and qPCR amplification was performed 0, 0.5, 1, 2, 4 and 8 hours. The level of PKCδ mRNA at each time point was calculated relative to untreated fibroblast cells and plotted on a semi-log scale. Exponential curve fitting was used to calculate the half-life from the slope of the curve using T½={−0.693/K} formula.

Adenoviral Vector Transfection

Adenoviral vectors containing green fluorescent protein (GFP, Ad-GFP), and dominant negative or wild-type PKCδ isoforms (Ad-dnPKCδ and Ad-wtPKCδ) were constructed and used to infect fibroblasts as described previously (Geraldes et al., Nat Med. 2009 November; 15(11):1298-306). Infectivity of these adenoviruses was evaluated by the percentage of green light-emitting cells under a fluorescent microscope (Nikon, Avon, Mass.). The presence of ˜80% of Ad-GFP-positive cells was considered to be a successful infection and used for further experimentation. Moreover, expression of each recombinant protein was confirmed by Western blot analysis, and expression was increased ˜4 to 8-fold with all constructs as compared with cells infected with controls adenovirus.

siRNA Transfection

The transfection of siRNA was performed using the Ambion Silencer Select Validated siRNA kit for primary cells (Ambion by Life Technology, Carlsband, Calif.), in 60-70% confluent fibroblast. For evaluating insulin's effect on VEGF production, cells were washed twice with PBS, and starved overnight with DMEM containing 1% BSA. Insulin (100 nM) was added to each for additional 16 h. After incubation, media were collected for VEGF measurement by ELISA and the cell lysate were collected for measuring protein concentration.

Histology and Immunohistochemical Analyses

Paraffin embedded sections were subjected to immunofluorescence staining using standard methods (Li et al., Circ Res. 2013 Aug. 2; 113(4): 418-427). Sections were incubated with antibodies (anti-CD31 (1:20); anti-Vimentin (5 ug/ml); anti-MHC Class 1 (1:250); anti-VEGF (1:100); or anti-PDGF BB (1:200) antibodies) or negative controls (0.1% BSA in 1× PBS), followed by incubation with fluorescent secondary antibody and staining the nuclei with DAPI as described before (Li et al., Circ Res. 2013 Aug. 2; 113(4): 418-427). Images were taken using Olympus FSX100 microscope.

Cell Migration Assay

Scratch wound migration assay—100% Confluent starved Medalist or control fibroblasts wounded, followed by incubation with 10 ng/ml PDGF-BB or 100 nM insulin for 12 hours. Phase contrast images of wounded areas were taken at time 0, 4, 6, 8 and 12 hours after stimulation, and migration was determined as described before (Ito et al., Am J Physiol Lung Cell Mol Physiol. 2014 Jul. 1; 307(1):L94-105).

Migration in Matrigel™ invasion chamber—Fibroblasts seeded at a density of 3×10⁴ cells/well into the upper chambers of Transwell inserts (BD Biosciences, Bedford, Mass.). The lower chambers were filled with medium containing 0.1% BSA with 10 ng/ml PDGF-BB. After 24 hours incubation, the number of migrated cells was counted in 10 random fields at 40× magnification under the microscope as described in Tancharoen et al., PloS one. 2015; 10(2):e0117775.

Cell Proliferation Assay

BrdU ELISA kit was used for the quantification of cell proliferation based on the measurement of BrdU incorporation according to the kit protocol (Abcam, Cambridge, Mass.) (Rui, PloS one. 2014; 9(12):e115140).

MicroRNA (miRNA)

Total RNA was isolated from primary human fibroblasts cells by the TRIzol method (Life Technologies, Grand Island, N.Y.), and quantified by Nanodrop-1000 (Thermo Scientific, Wilmington, Del.). 800 ng of total RNA was reverse transcribed using the miRNome MicroRNA Profiler kit following the manufacturer's protocol (System Biosciences, Mountain View, Calif.). Quantitative PCR was carried out in a Biorad CFX384 (Biorad, Hercules, Calif.). The non-coding RNA U6 was used for normalization of miRNA qPCR results.

Other Methods

Protein levels of VEGF in the medium were measured using Quantikine R&D System kit (Minneapolis, Minn.). This kit determines mainly VEGF165. Glycated hemoglobin (HbA1c) was determined by HPLC (Tosoh G7 and 2.2, Tokyo, Japan). Serum creatinine was determined by spectrophotometry. Urine albumin and creatinine were determined by turbidimetric methods. Serum C-peptide was determined by RIA (Beckman Coulter, Inc, Fullerton, Calif.).

Statistical Analysis

Univariate analyses were performed to determine distribution, the descriptive statistics are presented as appropriate using median [IQR], mean±SEM or mean±SD. Two way two-tailed t-test or chi-square tests were used for comparisons based on the distribution and number of observations. P-values less than or equal to 0.05 were considered statistically significant. STATA SE (College Station, Tex.) was used to perform all analyses.

### Example 1

### Clinical Characteristics of the Medalists and Non-Diabetic Controls

Fibroblasts were derived from 26 patients with long-standing (67.5±11 years) type 1 diabetes (T1D) (Medalists) with or without cardiovascular disease (CVD) and 7 age and gender matched donors without diabetes (controls). Table 1 shows a tabulated summary of the subject source of the fibroblasts, with their clinical characteristics and disease status (Table 1). Overall, participants have a mean age of 79 [64-76] and 76 [63-84] years in the Medalists and controls, respectively. Diabetes was diagnosed in the Medalists at 12 years [3-30] and had a mean duration of 67 years [51-85]. Their mean HbA1c was 7.2% [5.6%-9%], and have mean body mass index (BMI) of 24.8 [22.5-27.5]. Hypertension was found in 57.7% of the Medalists, defined as blood pressure of 135/85 mmHg or higher or use of anti-hypertensive medications.

Out of the 26 Medalist patients, 69% (18) reported a history of CVD (Table 1). Of the Medalists with CVD, 7 (39%; p=0.048) reported amputation (toe, below or above knee) and 4 (22%; p=0.2) reported lower extremity peripheral vascular disease. No Medalists without CVD reported amputation or peripheral vascular disease (Table 1). Estimated glomerular filtration rate (eGFR) of >45 ml/min/1.73 m² was found in 75% and 44% of the Medalists without or with CVD, respectively. eGFR of <45 ml/min/1.73 m² was found in 25% and 55% of Medalists without or with CVD, respectively. In Medalists without CVD, no-mild NPDR and PDR was observed in 6 (75%) and 2 (25%), respectively. In Medalists with CVD, 7 (39%) and 8 (44%) have NPDR and PDR, respectively. Neuropathy was reported in 25.0% and 66.6% (p=0.039) of Medalists without or with CVD, respectively.

TABLE 1

| | | | | Medalists | | |
|---|---|---|---|---|---|---|
| | All | | | without | Medalists | |
| | Medalists | Controls | p | CVD | with CVD | P |
| n | 26 | 7 | | 8 (30) | 18 (70) | |
| Age-yrs. [range] | 79.5 ± 8.9 | 76.00 ± 9.07 | 0.381 | 75.71 ± 13.31 | 81.39 ± 6.43 | 0.159 |
| | [56-93] | [63-84] | | [56-93] | [66-89] | |
| Male sex-no. (%) | 14 (53.8) | 5 (71.4) | 0.247 | 4 (50) | 10 (55.55) | 0.317 |
| Age of diagnosis-yrs. | 12.27 ± 7.84 | — | | 10.62 ± 6.70 | 13.00 ± 8.37 | 0.487 |
| [range] | [3-30] | | | | | |
| Duration-yrs. | 67.54 ± 11.11 | — | | 65.25 ± 10.22 | 68.56 ± 11.62 | 0.5 |
| | [51-85] | | | [52-77] | [51-85 | |
| HbA1C (%) | 7.27 ± 0.91 | — | | 7.10 ± 0.75 | 7.35 ± 0.98 | 0.527 |
| | [5.6-9] | | | | | |
| eGFR (ml/min/1.73 m\hat{ }2) | 51.38 ± 21.23 | — | | 62.60 ± 19.52 | 46.10 ± 20.40 | 0.072 |
| C-peptide (ng/mL) | 0.38 ± 0.57 | — | | 0.22 ± 0.13 | 0.47 ± 0.70 | 0.235 |
| BMI (kg/m\hat{ }2) | 24.83 ± 4.93 | — | | 26.71 ± 6.24 | 23.95 ± 4.11 | 0.280 |
| CVD-no. (%) | 18 (69) | — | | 0 | 18 (100) | |
| Nephropathy-no. (%) | | — | | | | |
| eGFR (ml/min/1.73 m\hat{ }2) ≥ | 14 (54) | — | | 6 (75) | 8 (44) | 0.061 |
| 45 | | | | | | |
| eGFR (ml/min/1.73 m\hat{ }2) < | 12 (46) | — | | 2 (25) | 10 (56) | 0.309 |
| 45 | | | | | | |
| Retinopathy-no. (%) | | — | | | | |
| NPDR | 13 (50) | — | | 6 (75.00) | 7 (39) | 0.085 |
| PDR | 10 (38) | — | | 2 (25.00) | 8 (44) | 0.23 |
| No report | 3 (12) | — | | 0 | 3 (17) | |
| Neuropathy-no. (%) | 14 (54) | — | | 2 (25) | 12 (67) | 0.039 |
| HTN-no. (%) | 15 (58) | 3 (43) | 0.26 | 2 (25) | 13 (72) | 0.031 |
| Skin ulcer-no. (%) | 8 (31) | — | | 2 (25.00) | 6 (33) | 0.332 |
| Any amputation-no. (%) | 7 (27) | — | | 0 | 7 (39) | 0.048 |
| Leg bypass/artery | 4 (15) | — | | 0 | 4 (22) | 0.204 |
| angioplasty-no. (%) | | | | | | |

Median + SD [Q1, Q3] or (%). Chi-square or fisher's tests were used to compare continuous variables between the two groups.
CVD = cardiovascular disease,
HTN = hypertension,
BMI = body mass index,
NPDR = non proliferative diabetic retinopathy,
PDR = proliferative diabetic retinopathy,
eGFR = estimated glomerular filtration rate,
HbA1c = Glycated hemoglobin

### Example 2

### Effect of Glucose, Insulin, and Hypoxia on VEGF Expression

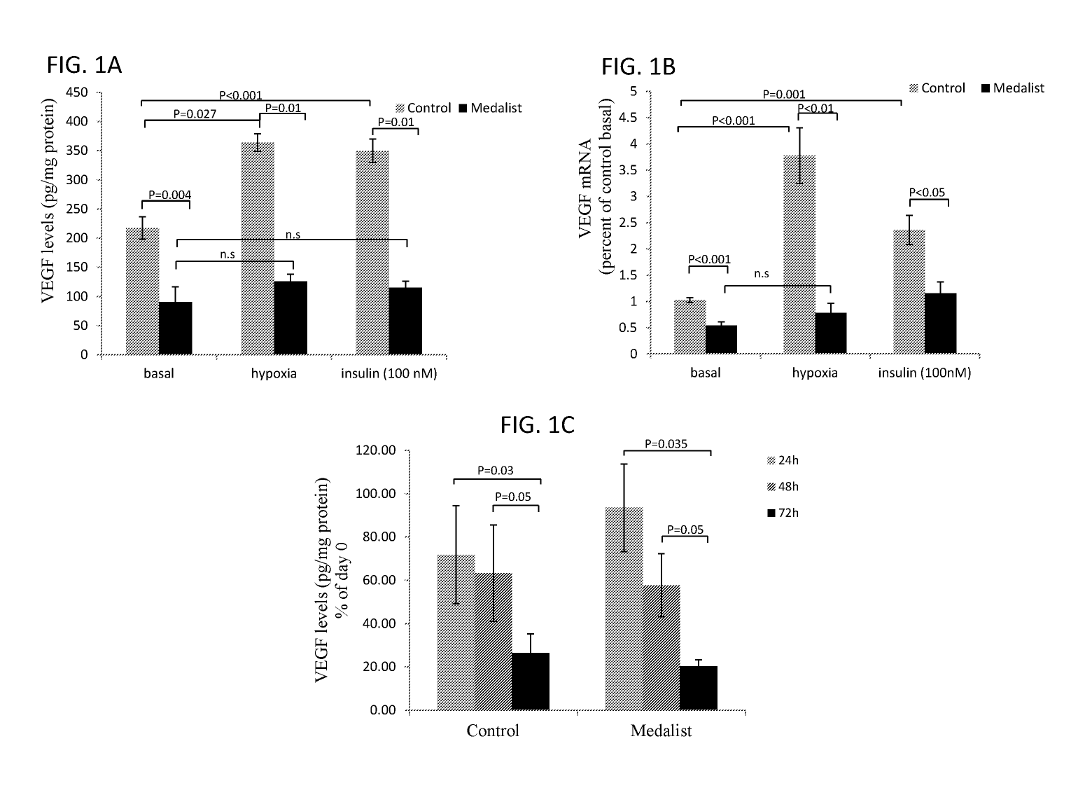

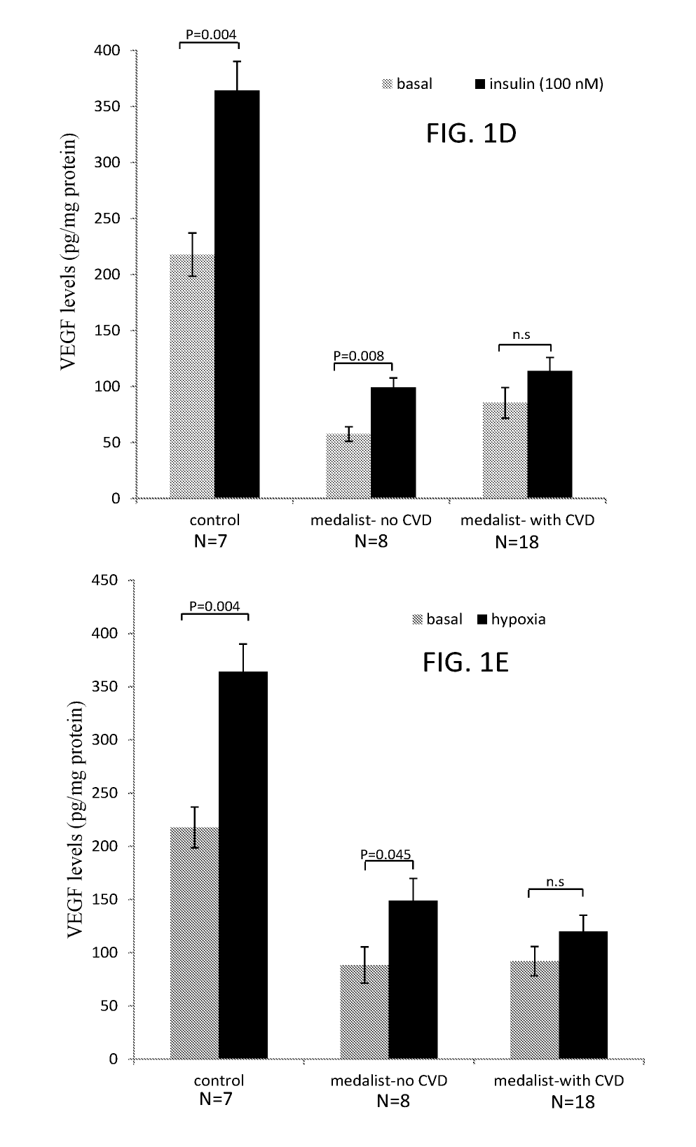

Basal VEGF protein secretion (FIG. 1A) and mRNA levels (FIG. 1B) were lower in fibroblasts of Medalists than in fibroblasts of controls (95.5±26 vs. 210.7±19.3 pg/mg protein, p=0.004; and 0.50±0.07% vs. 1.00±0.05, p<0.001, respectively).

Similarly, in fibroblasts from both controls and Medalists incubated with 25 mM glucose for 3 days, VEGF protein production was significantly reduced (24 hrs: 71.8±22.7%, 48 hrs: 63.3±22.2%, 72 hrs: 26.5±8.7% of day 0 at 5.6 mM glucose in control cells and 93.3±20.2%, 57.7±14.6%, 20.3±3.0% of day 0 at 5.6 mM glucose in Medalist cells) (FIG. 1C). Hypoxia and insulin stimulation increased VEGF protein levels by 77% (p=0.027) and by 66% (p<0.001), respectively, and VEGF mRNA by 3.8 and 2.4-fold (p=0.001), respectively, in fibroblasts from controls. However, in fibroblasts from Medalists, both hypoxia and insulin failed to significantly stimulate VEGF protein (FIG. 1A) and mRNA production (FIG. 1B). Interestingly, separating the Medalists according to the presence of chronic complications status revealed a significant increase in VEGF protein levels in response to insulin in the Medalists without CVD compare to Medalists with CVD (57.5±8.5 or 75.5±13.7 pg/mg protein in the basal and 99.1±8.4 or 106.0±12.0 pg/mg protein after insulin stimulation in Medalists without CVD or Medalists with CVD, respectively) (FIG. 1D). Similarly, hypoxia significantly increased VEGF protein levels in the Medalists without CVD compare to those with CVD (88.4±17.0 or 92.1±13.8 pg/mg protein at basal and 148.8±20.9 or 119.8±15.6 pg/mg protein in hypoxic condition in Medalists without CVD or Medalist with CVD, respectively) (FIG. 1E).

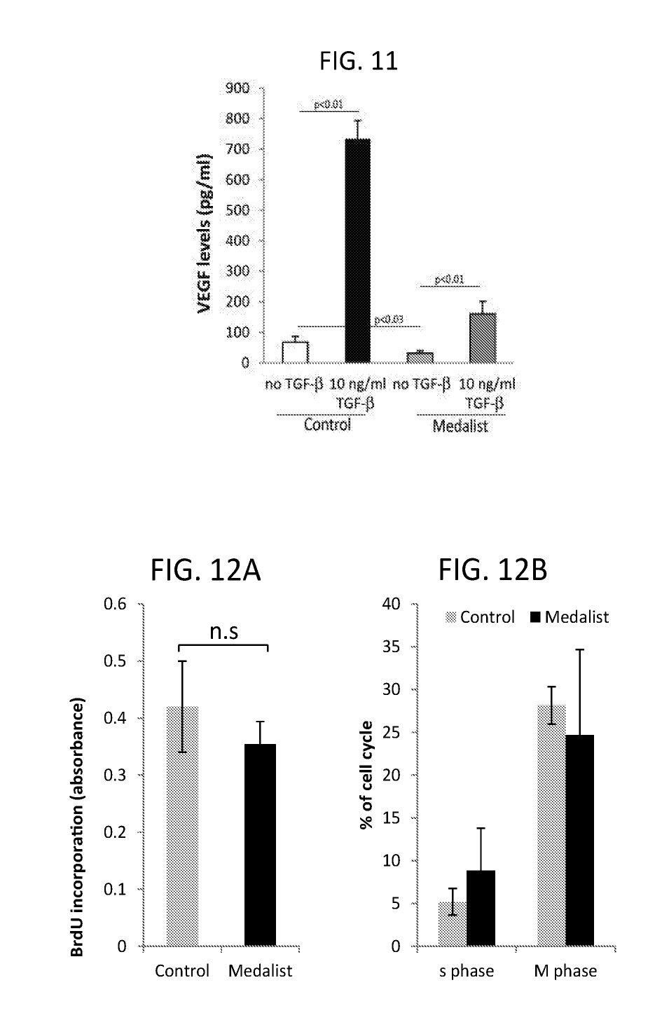

Stratifying the Medalists by neuropathy also revealed a differential response to insulin stimulation; the response was lower in fibroblasts from patients with neuropathy than in fibroblasts from patients without neuropathy (FIG. 10A). Differences were not seen in responses to insulin or hypoxia stimulation for other strata of diabetes complications (FIGS. 10B and 10C). In contrast to the finding of reduced VEGF secretion in the Medalist fibroblasts compare to the controls, TGF-β expressions in the fibroblasts of both groups were not different (data not shown). Furthermore, we have evaluated the effect of TGF-β to induce VEGF expression and secretion into the media. The results clearly showed that VEGF induced by TGF-β was blunted in fibroblasts from Medalists compared to controls (FIG. 11).

### Example 3

### Effect of Glucose or Growth Factors on Fibroblast Migration and Proliferation

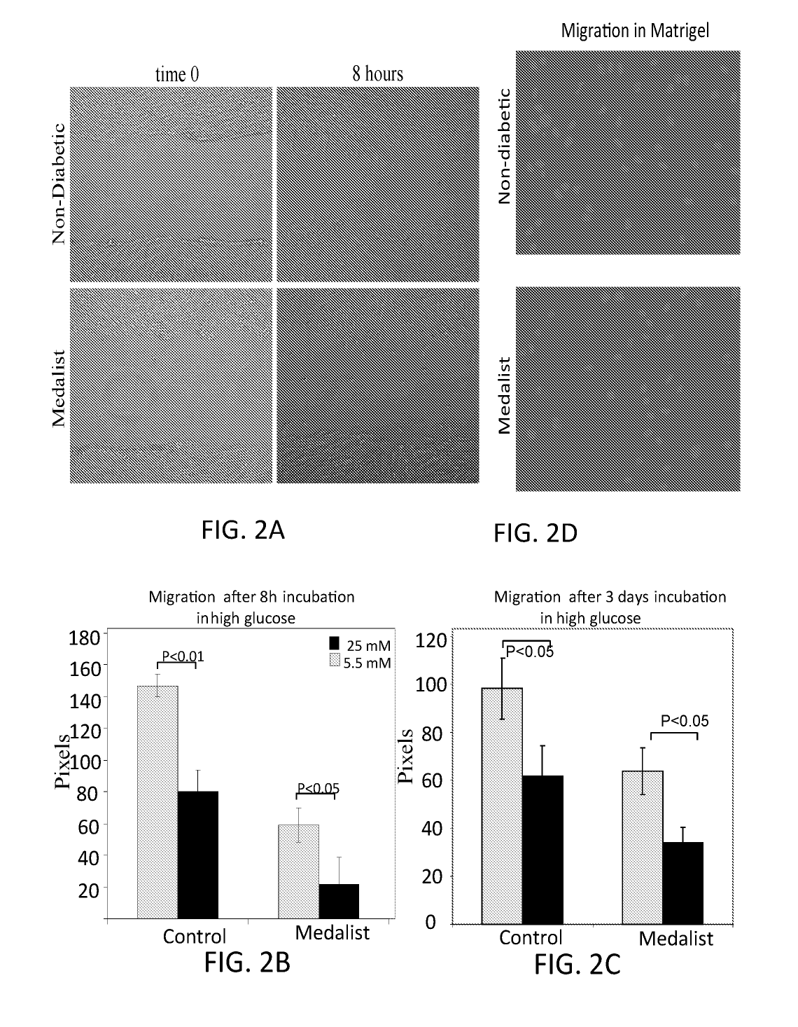

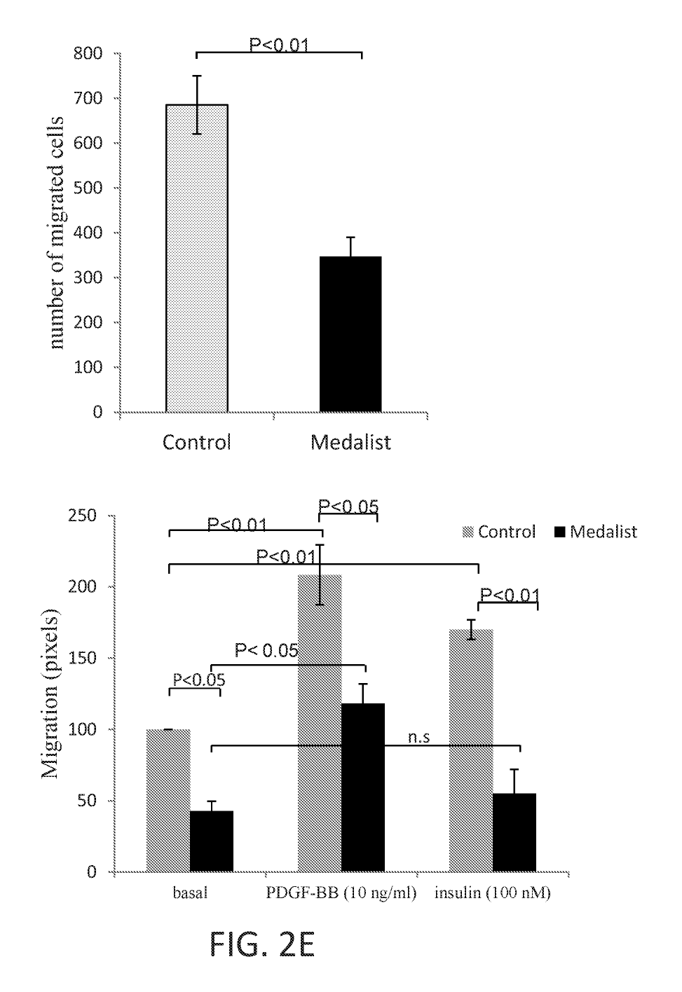

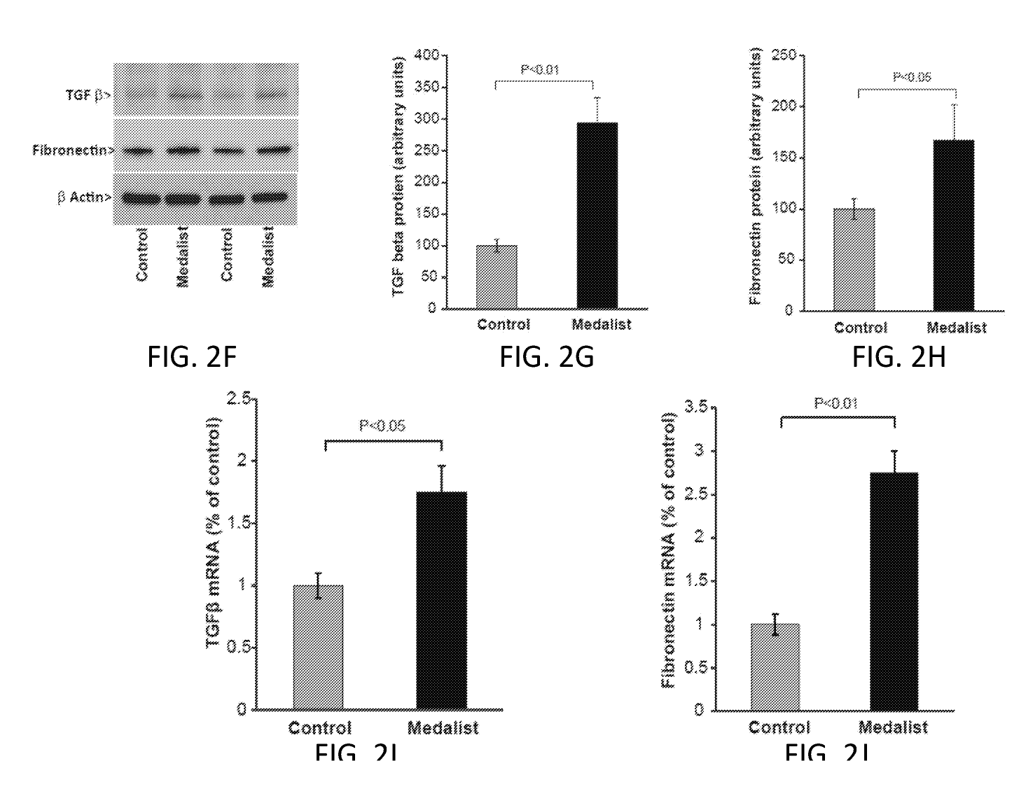

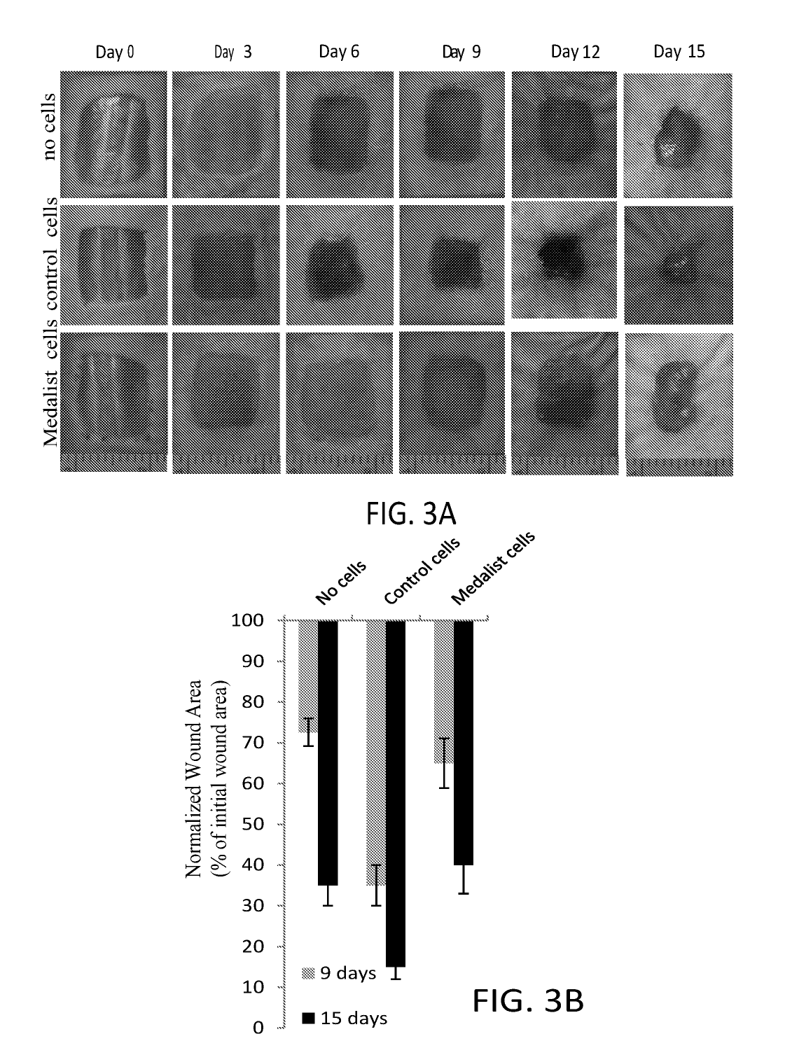

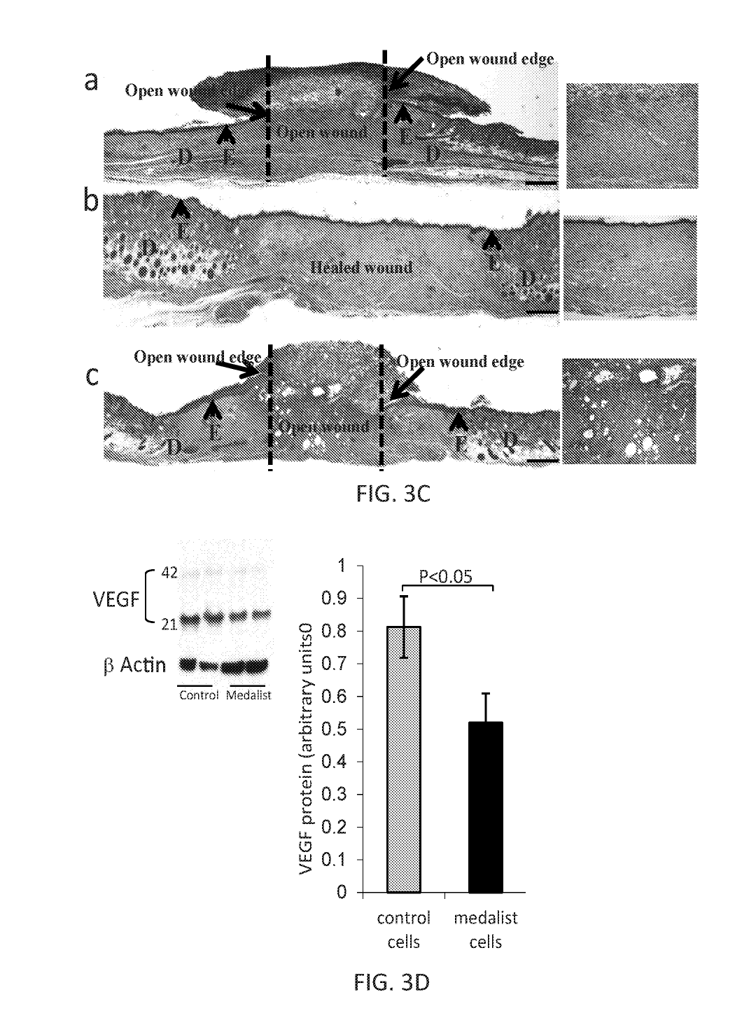

In Medalists compared to controls, less fibroblast migration was observed, as measured by the scratch assay: (59±11 vs. 147±7pixels, p<0.01) (FIGS. 2A-C); and less cell migration in Matrigel chambers: 347±43 vs. 685±65 migrated cells, p<0.01 (FIG. 2D). Furthermore, incubation of fibroblasts of both controls and Medalists with 25 mM glucose for either 8 h (FIG. 2B) or 3 days (FIG. 2C) revealed significantly decreased fibroblast migration. PDGF-BB increased cell migration significantly in fibroblasts of both controls and Medalists (FIG. 2E). However, insulin increased cell migration in control fibroblasts by 1.7-fold (p<0.05) but failed to significantly increase cell migration in Medalist fibroblasts (FIG. 2E). TGFβ and fibronectin protein expressions (FIGS. 2F, G, and H) and TGFβ and fibronectin mRNA levels (FIGS. 2I-J) were increased in the Medalist fibroblasts compared to those of controls [293.5±40.0 vs. 100±10 arbitrary units (au) (p<0.01); 167±35 vs. 100±10 au (p<0.05); 1.75±0.25 vs. 1.0±0.1-fold (p<0.05); 2.8±0.4 vs. 1.0±0.1-fold (p<0.01), respectively]. Fibroblast proliferation, as determined by bromodeoxy uridine (BrdU) incorporation (FIG. 12A) or by flow cytometry (FIG. 12B), exhibited no significant difference between Medalists and controls (0.35±0.04 vs. 0.42±0.08%, 8.9±2.2 vs. 5.2±1.5% in S phase and 24.7±10.0% vs. 28.2±4.9% in M phase, respectively).

### Example 4

### Medalists Fibroblast Display Impaired Wound Healing In Vivo

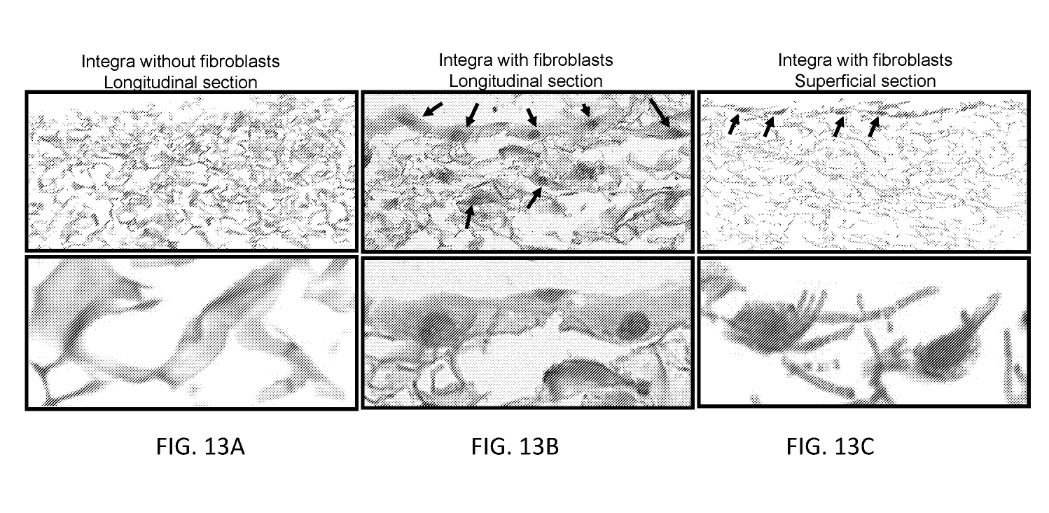

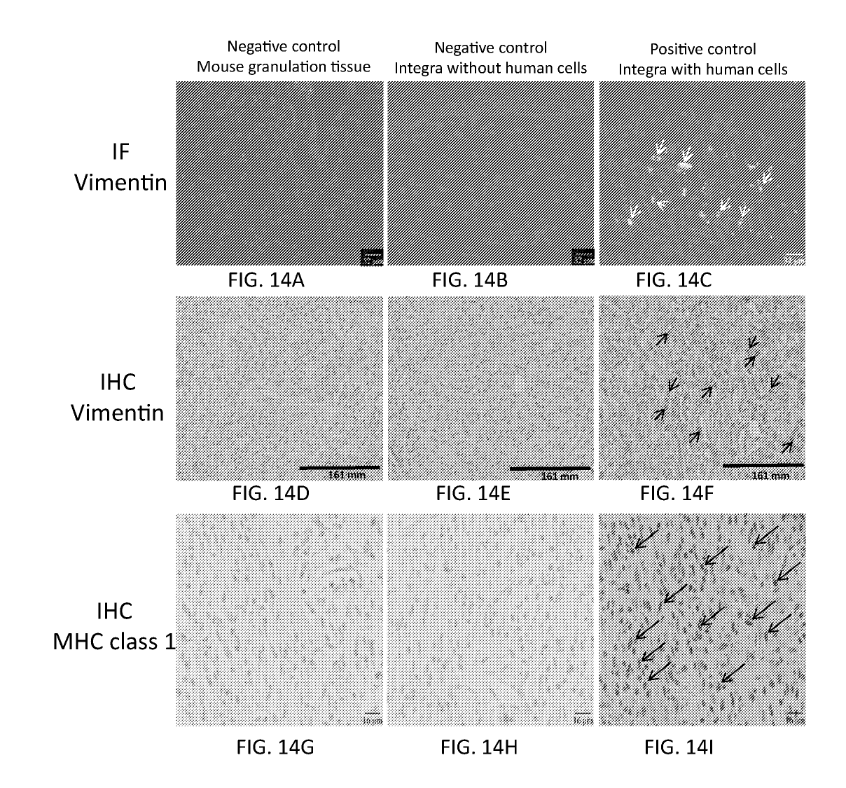

To investigate the functional properties of fibroblasts in wound repair, an Integra dermal regeneration template, consisting of a collagen-glycosaminoglycan (GAG) scaffold bilayer matrix wound dressing, was used to transfer human adenoviral vectors containing fibroblasts labeled with green fluorescent protein (GFP), from controls and Medalists to a dorsal full thickness cutaneous wound model in nude mice. For controls and Medalists, the presence of human fibroblasts on Integra before transplantation was confirmed by H&E staining (FIGS. 13A-C) and GFP labeled cells. Characterization of fibroblasts on Integra in the wound granulation tissues at 9 days after transplantation was demonstrated by immunohistochemistry for human vimentin (FIGS. 14D-F), MHC class 1 (FIGS. 14G-I), and immunofluorescence for human vimentin (FIGS. 14A-C).

Macroscopically, wound areas were quantitated by the proportion of the wound surface not covered by an epithelial layer, divided by the original wound area. In experiments using control fibroblasts, the wound area was 35% on day 9, and 15% on day 15 (FIG. 3A-B), contrasting with 65% and 60%, respectively, in experiments using Medalist fibroblasts (FIGS. 3A and B). The efficiency of wound healing was assessed by measuring at 9, 12, and 15 days post-initial wound, the distance between bi-lateral edges of granulation tissues consisting of newly formed capillaries, fibroblasts, and macrophages, as stained by H&E (FIG. 3C). Amongst the specimens with transplanted control cells on the Integra membrane, the entire granulation area was completely healed by day 15, compared with healing of only 60% (p<0.05) in specimens with Medalist cells (FIG. 3C).

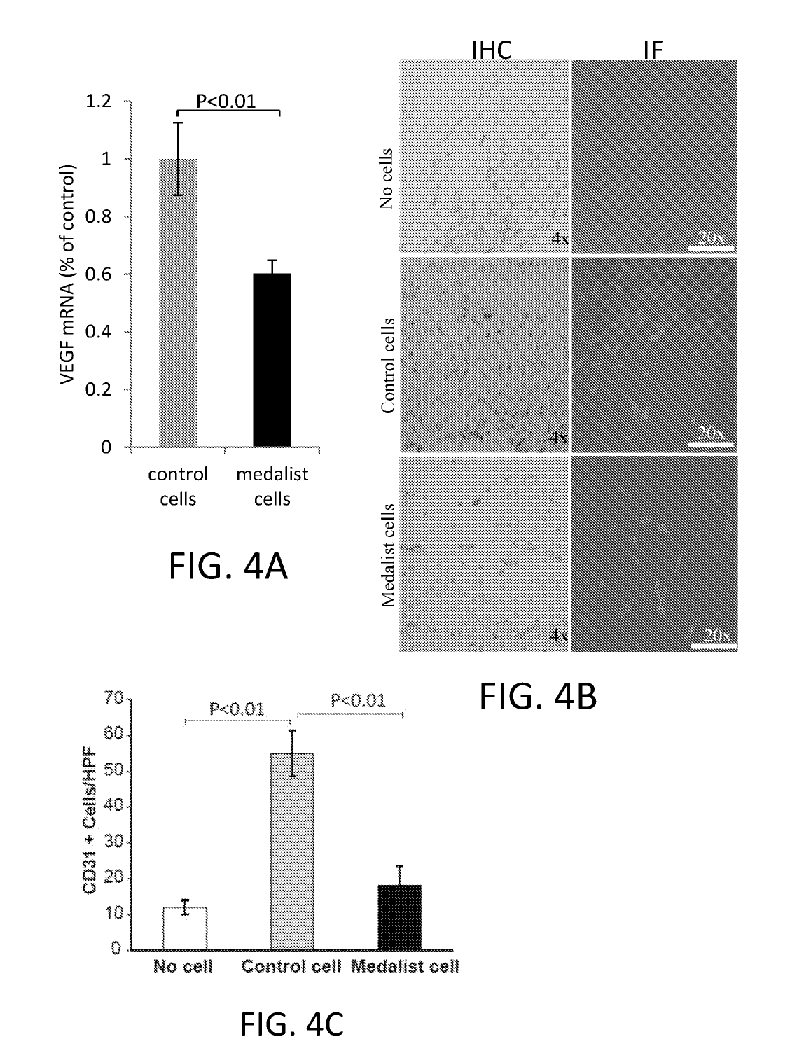

Protein and mRNA levels of VEGF were 56% (p<0.05) and 65% (p<0.01) lower on day 15 post-wounding in granulation tissues transplanted with Medalist fibroblasts than fibroblasts from controls (FIGS. 4A and B). These results were supported by immunohistochemistry data showing reduced VEGF and PDGF-BB expressions in the granulation tissue transplanted with Medalist fibroblasts compared to fibroblasts from controls. When assessed by CD31+ positive cells, the extent of neovascularization in granulation tissues was 3-fold greater (p<0.01) in wounds with control vs. Medalist fibroblasts (FIG. 4C and quantification in FIG. 4D).

### Example 5

### Assessing Insulin Signaling in the Controls and Medalist Fibroblast

Since fibroblasts from Medalists exhibited abnormal VEGF expression and migration in response to insulin stimulation, insulin signaling was characterized to identify the specific step of abnormality in the signaling pathway. Basal p-AKT (Ser473) and p-ERK (Thr202/Tyr204) expression were respectively 30% (non-significant) and 42% (p<0.05) higher in fibroblasts of Medalists than in those of controls (FIGS. 5A-D). Insulin-stimulated p-AKT increased by 3.6-fold in control fibroblasts (p<0.01) and by 2-fold (60% net, p<0.05) in Medalist fibroblasts, compared to untreated cells (FIGS. 5A-D). Conversely, PDGF-BB increased p-AKT by 7-fold in both control and Medalist fibroblasts (FIGS. 5A and B). Insulin and PDGF-BB stimulation of p-ERK were similar in both groups (FIGS. 5C and D).

Responses to insulin stimulation (100 nM) were compared in fibroblasts from Medalists with and without CVD. Insulin significantly increased p-AKT by 1.8-fold (p<0.05) and by 2.4-fold (p<0.01) in fibroblasts of Medalists with and without CVD respectively, which was lower than the 4.0-fold increase of p-AKT following insulin stimulation in the control fibroblasts (p<0.01) (FIG. 5E). Insulin-induced IRS1 activation in tyrosine phosphorylation at site 649 (p-Tyr649) was increased by 53% (p<0.01), 34%, and 63% (p<0.05); and at site 911 (p-Tyr911) by 52% (p<0.05), 26%, and 40% (p<0.05) in fibroblasts of controls, Medalists with CVD, and Medalists without CVD, respectively. This illustrates significantly lower activation in fibroblasts of Medalists with CVD than in fibroblasts of Medalists without CVD (FIGS. 5E-H). Surprisingly, insulin-stimulated levels of p-Tyr of the insulin receptor beta subunit were all similarly increased 5.2-, 4.7-, and 4.9-fold in controls, Medalists with CVD, and Medalists without CVD, respectively (p<0.01) (FIGS. 5E and F).

### Example 6

### Evaluation of Protein Kinase C Activation in the Fibroblast and Granulation Tissues

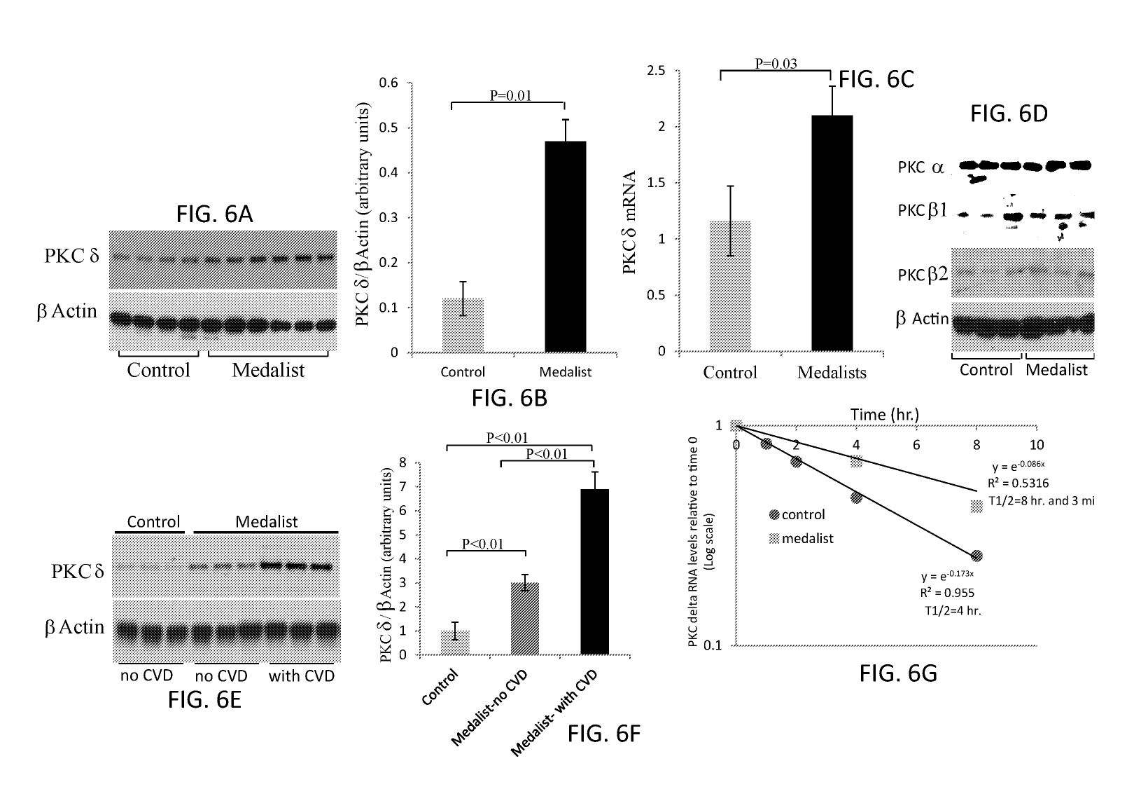

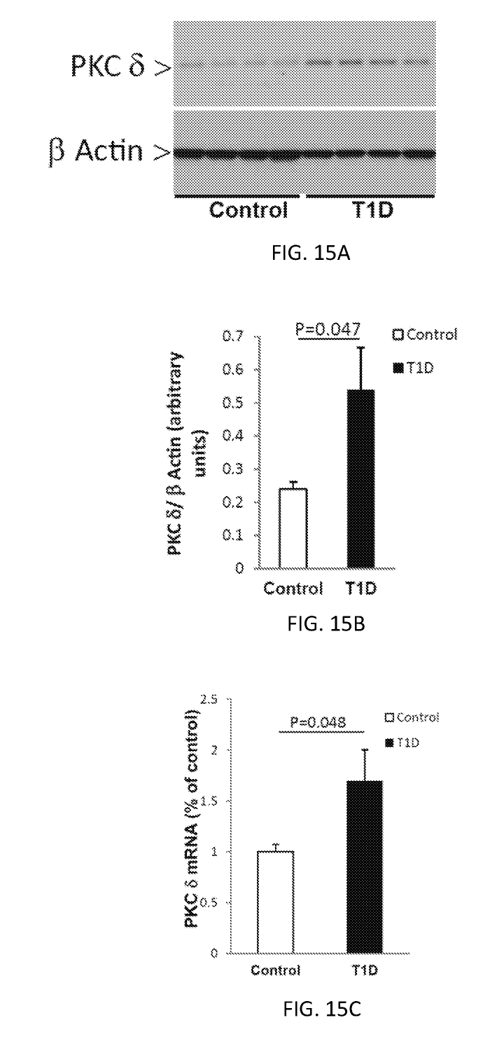

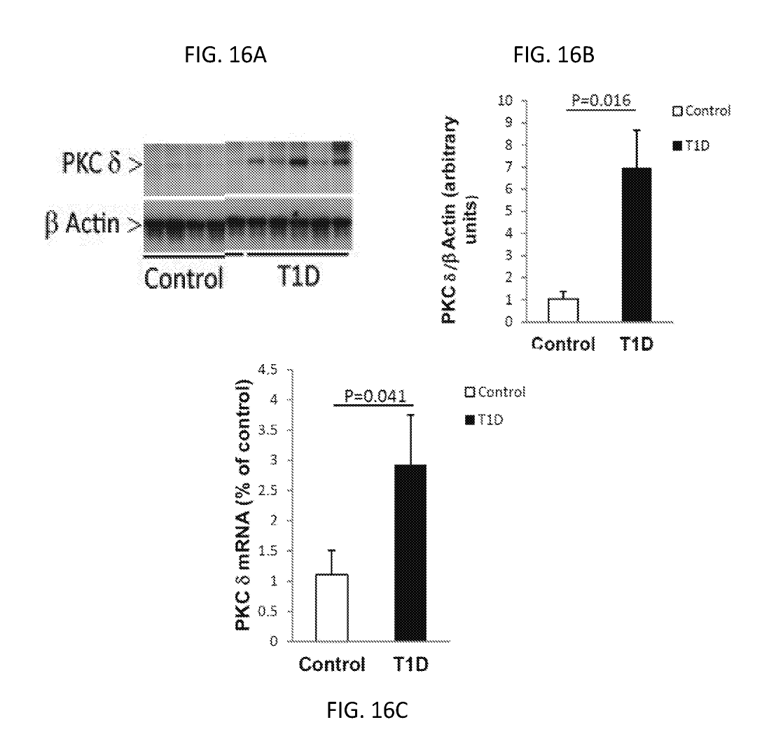

Activation of the PKC family has been reported to inhibit the insulin signaling pathway and contribute to the development of diabetic complications (Geraldes et al., Circulation research. 2010; 106(8):1319-31). To evaluate the role of PKC activation on the reduction of insulin signaling and VEGF secretion, PKC isoform expression and activation in fibroblasts from controls and Medalists were assessed. Levels of PKCδ protein and mRNA expression were increased significantly in fibroblasts from Medalists compared to fibroblasts from controls (3.8- (p=0.01) and 2-fold (p=0.03), respectively) (FIGS. 6A-C), without significant changes in PKCδ, PKCβ1, and PKCβ2 protein expressions (FIG. 6D). The increase in PKCδ protein expression was more prominent in Medalists with CVD than in Medalists without CVD: 7- vs. 3-fold (p<0.01; FIGS. 6E and F). Similar to our finding in post-mortem fibroblasts, PKCδ protein and mRNA were increased by 3 fold and 70%, respectively, in fibroblasts derived from living T1D patients compared to living control (FIGS. 15A-C). Furthermore, PKCδ protein and mRNA levels were increased by 7 and 3 fold, respectively, in discarded tissues obtained from active diabetic foot ulcers compared to control tissues (FIGS. 16A-C).

To determine whether the increase in PKCδ mRNA levels in Medalist fibroblasts is due to post-transcriptional regulation, a PKCδ mRNA stability assay was done. The half-life of PKCδ in RNA was analyzed by incubating cells with or without actinomycin-D (5 ug/ml) for 0-8 hours, followed by qRT-PCR analysis. PKCδ mRNA half-life in control fibroblasts was 4 hours and in Medalist cells, 8 hours, indicating increased PKCδ mRNA stability in the Medalist cells (p<0.05, FIG. 6G).

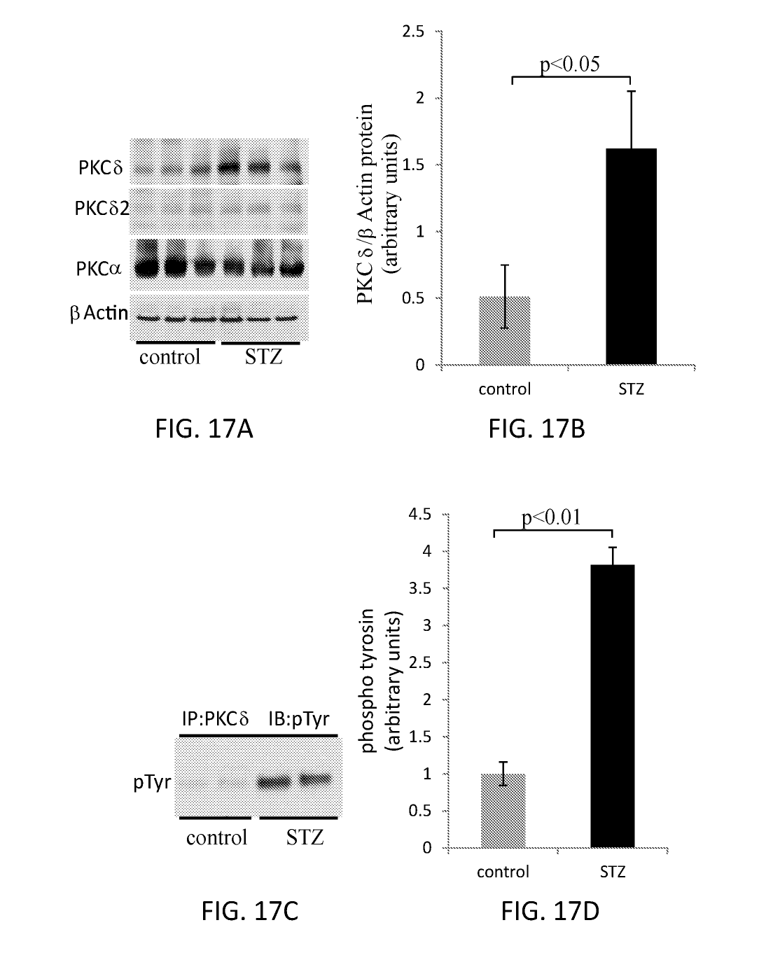

To confirm these observations regarding the elevation and activation of PKCδ isoforms in the wounds of diabetic models, granulation tissues were extracted from excision wounds obtained from STZ-induced insulin deficient diabetic mice. Two weeks after STZ injection, animals with fed blood glucose levels above 400 mg/dL were selected. Granulation tissue obtained 9 days after the initial wounding incision showed a 3.1-fold (p<0.05) increase in PKCδ protein expression (FIGS. 17A and B), and a 3.8-fold (p<0.01) increase in tyrosine phosphorylation of PKCδ after immunoprecipitation with anti-PKCδ antibody, a marker of PKCδ activation (Kikkawa et al., Journal of biochemistry. 2002; 132(6):831-9) (FIGS. 17C and D).

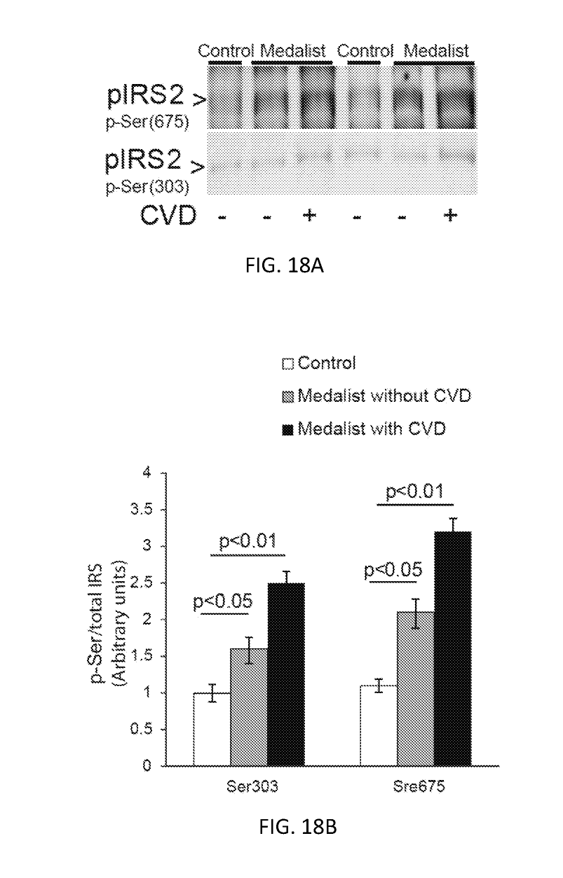

We recently identified serine phosphorylation sites at positions 303 and 675 on IRS2, which can be induced by PKC activation, and inhibit insulin-induced p-Tyr sites on IRS2 (positions 653 and 911), and its downstream signals such as p-AKT (Li et al., Circulation research. 2013; 113(4):418-27). To further confirm the inhibitory effect of PKCδ overexpression on insulin signaling on p-Tyr649 and p-Tyr911 of IRS2 in the Medalist fibroblasts, p-Ser sites of IRS2 were studied. Greater elevation of p-Ser303 and p-Ser675 were observed in fibroblasts from Medalists with CVD, from Medalists without CVD, and from control fibroblasts (2.5 vs. 1.6 vs. 1.0-fold for p-Ser303, 3.2 vs. 2.1 and vs. 1.0-fold for p-Ser675) (FIG. 18A-B).

### Example 7

### Effect of PKCδ Inhibition or Knockout on Insulin's Induce VEGF Secretion

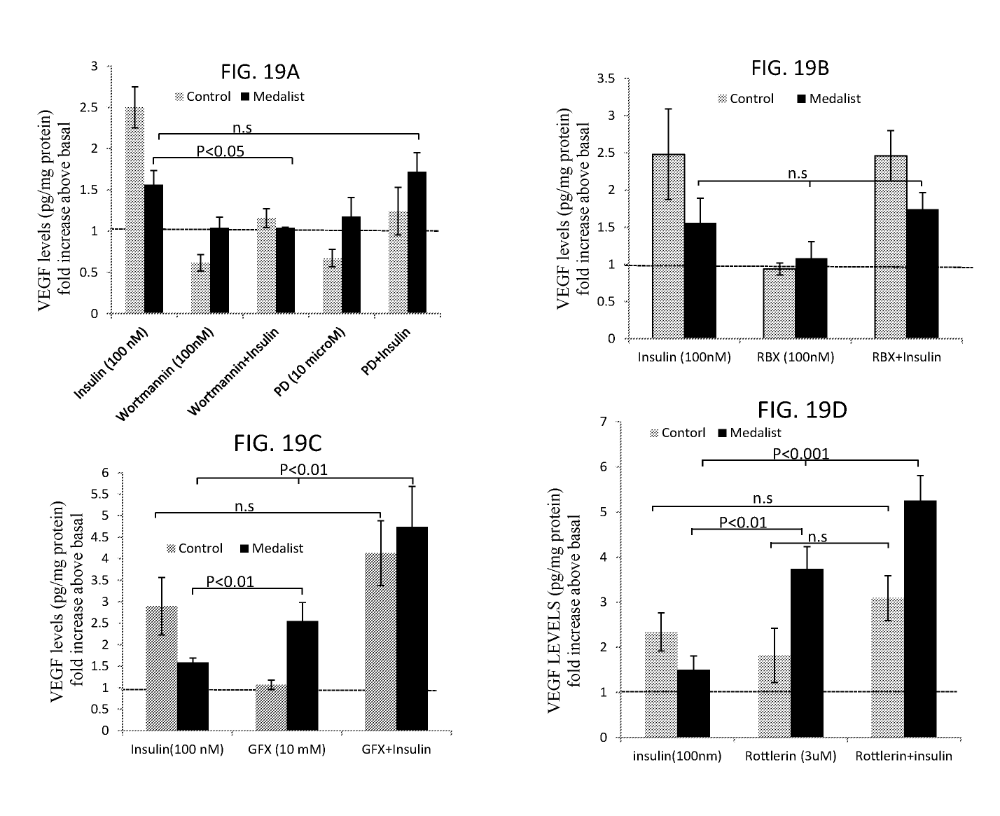

Our data suggest increased PKCδ expression, and activation inhibited insulin signaling in Medalist fibroblasts, resulting in decreased VEGF secretion and delayed wound healing in vivo. Thus, we examined whether inhibition of several intracellular signaling pathways mediated insulin signaling. We examined how PI3 kinase (wortmanin), MAP kinase (PD98059), general PKC (GFX), PKCβ (RBX), and PKCδ (rottlerin) affect insulin's induction of VEGF production in vitro (FIGS. 19A-D). Wortmanin, but not PD98059, significantly inhibited insulin-stimulated VEGF production in Medalist fibroblasts (p<0.05, FIG. 19A). Furthermore, treatment with RBX, a selective PKCβ isoform inhibitor, failed to increase insulin stimulated VEGF secretion (FIG. 19B). However, treatment with either GFX (FIG. 19C) or with 3 uM rottlerin (FIG. 19D) increased insulin-stimulated VEGF secretion in Medalist fibroblasts (insulin vs. GFX vs. GFX+insulin: 1.59±0.10 vs. 2.55±0.43 vs. 4.74±0.94-fold increase in VEGF levels above basal, p<0.01; and insulin vs. rottlerin vs. rottlerin+insulin: 1.50±0.31 vs. 3.74±0.49 vs. 5.25±0.56-fold increase in VEGF levels above basal, p<0.01) (FIGS. 19C-D).

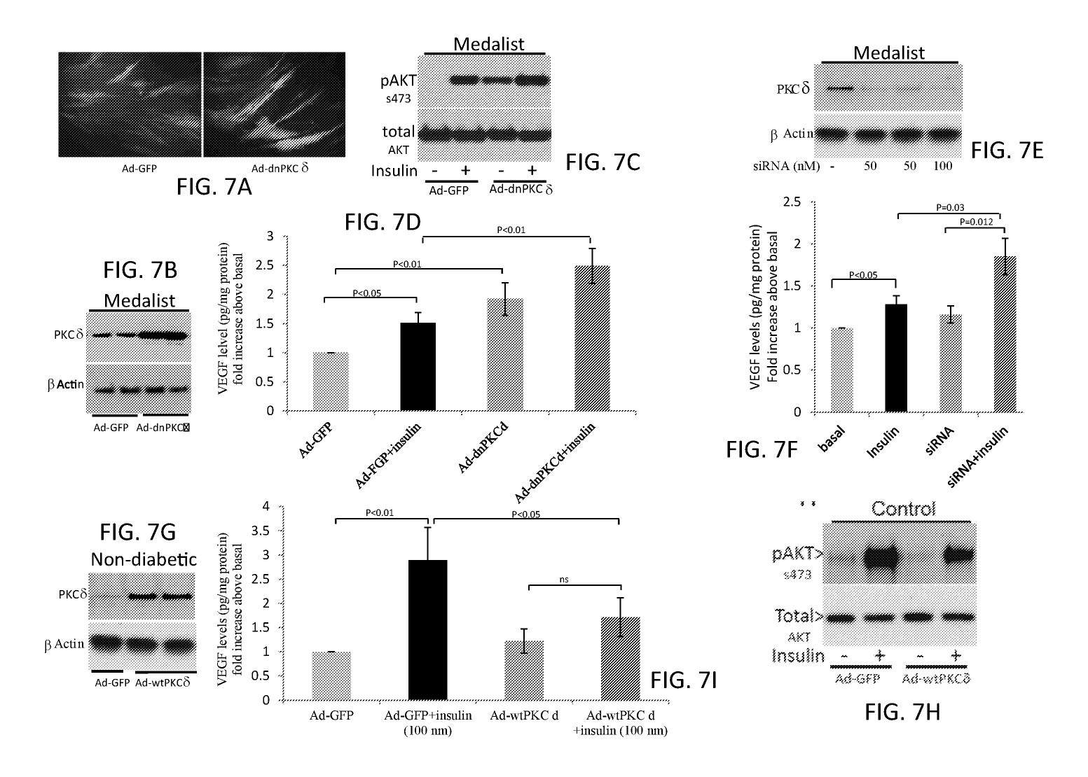

To specifically confirm that increased PKCδ expression in Medalist fibroblasts decreases insulin-stimulated VEGF secretion and delays wound healing in vivo, PKCδ expression in fibroblasts from Medalists was knocked down with siRNA or adenoviral vector infection with dominant negative PKCδ (Ad-dnPKCδ). Inhibition of PKCδ in Medalist fibroblasts with Ad-dnPKCδ resulted in increased insulin-stimulated p-AKT (FIG. 87B-C) and insulin-stimulated VEGF secretion (1.51±0.18 vs. 1.00±0.05-fold increase in Ad-GFP with and without insulin, p<0.05; and 2.79±0.30 vs. 1.92±0.28-fold increase in Ad-dnPKCδ with and without insulin, p<0.05) (FIG. 7D). Similarly, siRNA PKCδ knockdown in the Medalist fibroblasts increased insulin-stimulated VEGF secretion (siRNA, and siRNA with insulin: 1.28±0.10 vs. 1.16±0.10 and 1.85±0.21-fold increase above basal with insulin, respectively; p<0.05) (FIGS. 7E-F). However, increasing PKCδ expression in control fibroblasts by infection with Ad-wtPKCδ decreased insulin-stimulated p-AKT (FIG. 7H) and inhibited insulin-stimulated VEGF production compared to control Ad-GFP infected cells (without insulin 1.00±0.05 vs. with insulin 2.89±0.67-fold p<0.01; Ad-wtPKCδ without insulin:1.22±0.25 vs. with insulin:1.71±0.40-fold, p=ns) (FIGS. 7G-I).

### Example 8

### In-Vivo Knockout of PKCδ in Diabetic Fibroblast Improve Wound Healing Where Increasing PKCδ Expression in Control Fibroblast Delay Wound Healing

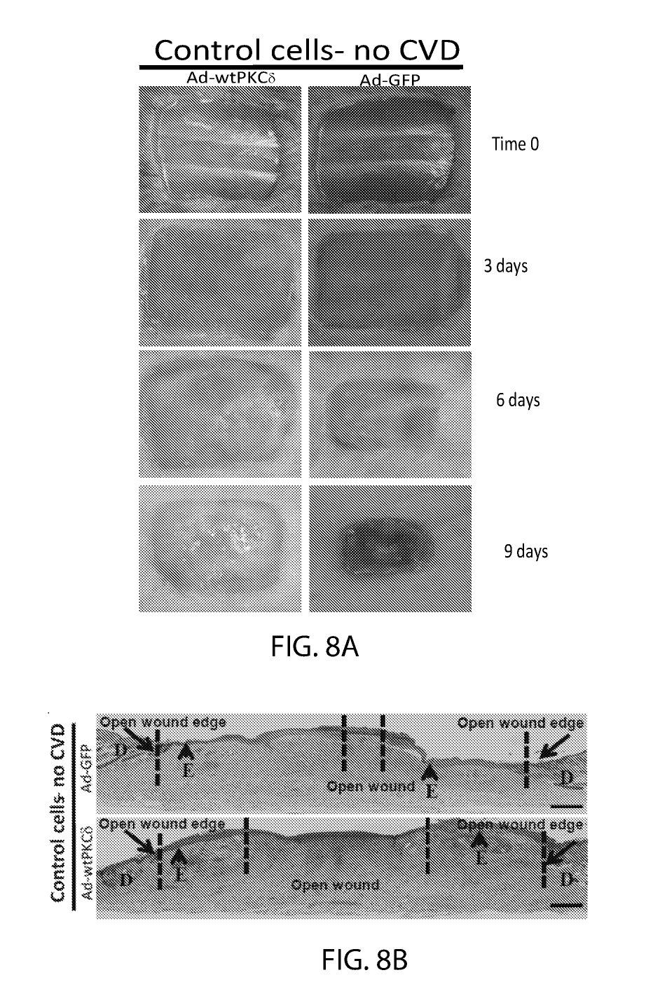

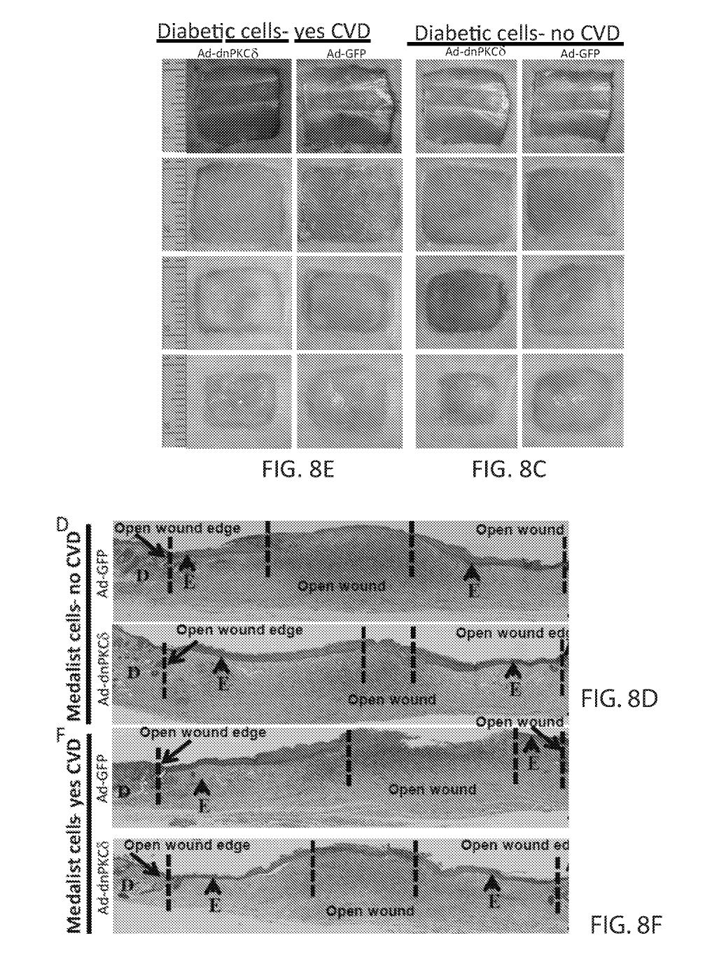

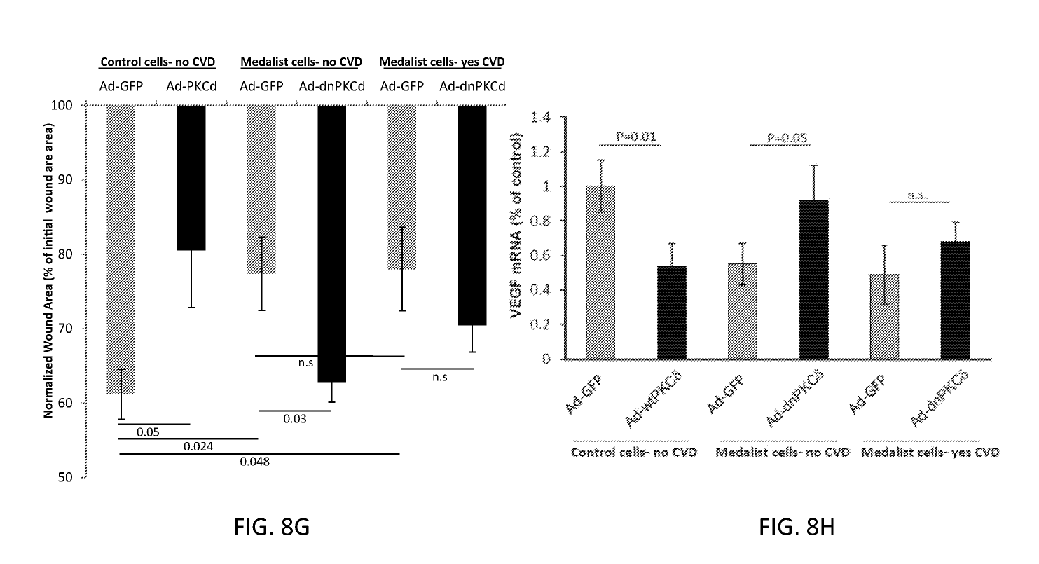

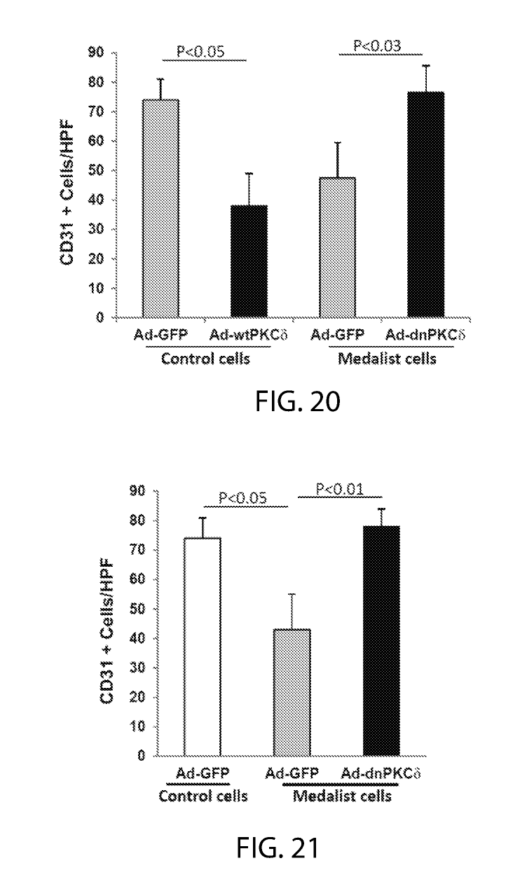

Control fibroblasts infected with Ad-wtPKCδ transplanted into control nude mice triggered significant delay in wound closure (61.1±3.3 vs. 80.5±7.6% of initial wound area in Ad-wtPKCδ vs. Ad-GFP infected cells, p=0.05) (FIGS. 8A, B, and G). Furthermore, transplants of fibroblasts from Medalists without CVD into control nude mice after knockdown of PKCδ significantly improved wound healing (Ad-dnPKCδ vs. Ad-GFP infected cells: 62.8±2.7% vs. 77.4±4.9% of initial wound area, p=0.03); compared to 70.4±3.5 vs. 78.0±5.6 in fibroblasts from Medalists with CVD (FIGS. 8C-G). The rescue experiments with knockdown of PKCδ in the Medalists fibroblasts' resulted in more neovascularization than in the untreated Medalists cells, as demonstrated by 2 fold increases in CD31+ positive cells in granulation tissues from non-diabetic mice (FIG. 20).

Additionally, to evaluate the effect of the diabetic milieu on wound healing in vivo, the procedures were repeated using STZ-induced diabetic nude mice yielding an insulin deficient model (FIG. 9). Ad-GFP infected control fibroblasts transplanted into diabetic mice resulted in a 60.0% and 79.6% closure of the initial wound area after 9 and 15 days, respectively (FIG. 9A-C). However, transplants of Ad-dnPKCδ infected Medalist fibroblasts in nude mice resulted in 78.4% and 92.3% closure of the initial wound area after 9 and 15 days, respectively (FIG. 9A-C). Transplants of these fibroblasts also normalized VEGF mRNA in wound granulation tissue (FIG. 9D). However, transplants of control Ad-GFP infected Medalist fibroblasts failed to improve wound closure (35% or 45% of initial wound area after 9 and 15 days, respectively) (FIG. 8A-C) or VEGF expression in wound granulation tissues (FIG. 9D). Knockdown of PKCδ expression in the Medalists fibroblasts' resulted in more neovascularization than in the untreated Medalists cells, as demonstrated by almost two fold increases in CD31+ positive cells in granulation tissues even in STZ induced diabetic mice (FIG. 21). The cells observed in the open wound area in FIGS. 8B and 9B are exudate and inflammatory cells as part of the granulation tissue.

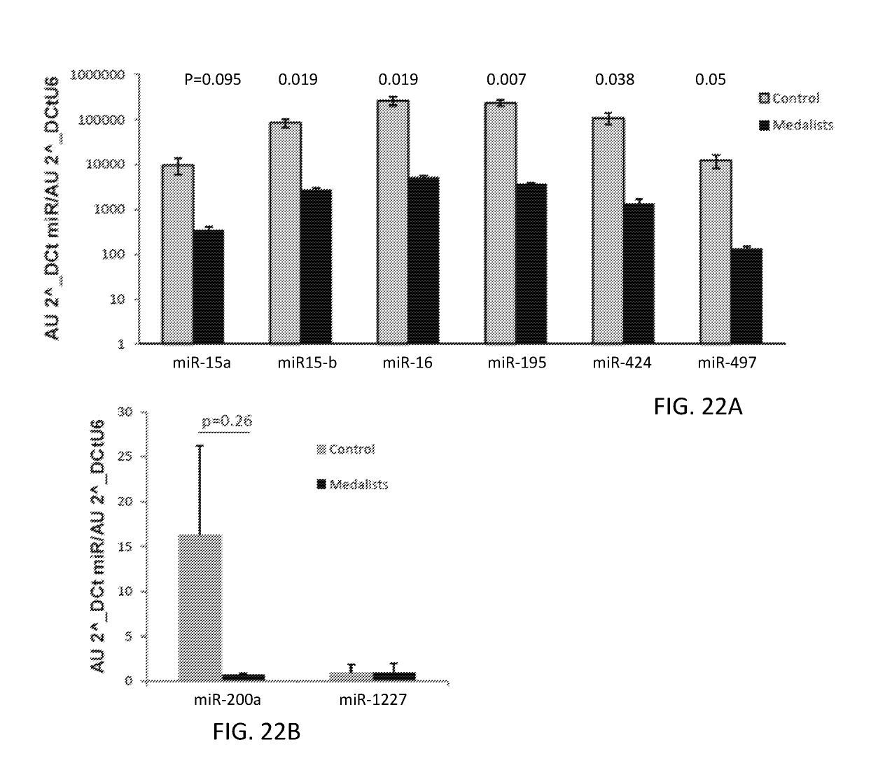

Additional experiments were performed to deepen our understanding of the mechanism for the persistent upregulation of PKCδ mRNA and protein in the Medalists' fibroblasts as presented in FIG. 6. To identify whether miRNAs might be involved in PKCδ expression in the Medalists, we assessed which miRNAs might be predicted to bind to the 3′-UTR of the PKCδ mRNA and were expressed differentially between Medalists and controls (see Table 2). The expression levels of the predicted interacting miRNAs were studied in the Medalists' fibroblasts compared to the controls using qPCR analysis. Interestingly, predicted PKCδ miRNA regulators miR-15a, 15b, 16, 195, 424, and 497 were significantly decreased in the Medalists compared to the controls (FIGS. 22A-B). In contrast, the expression levels of control miRNAs that were not predicted to regulate PKCδ, miR-1227 and miR-200a, did not differ between fibroblasts from Medalists and controls.

TABLE 2
miRNAs predicted to bind to the 3′-UTR of the PKCδ
mRNA in the Medalists and controls fibroblasts,
Predicted pairing of target region and miRNA

| | | SEQ |
|---|---|---|
| | | ID |
| Description | Sequence | NO: |
| Position 88-94 | 5′ GACUGUGGUGACUUCUGCUGCUG | 16 |
| of PKCδ 3′ UTR | | |
| (Target region) | | |
| hsa-miR-15a | 3′ GUGUUUGGUAAUACACGACGAU | 17 |
| hsa-miR-15b | 3′ ACAUUUGGUACUACACGACGAU | 18 |
| hsa-miR-16 | 3′ GCGGUUAUAAAUGCACGACGAU | 19 |
| hsa-miR-195 | 3′ CGGUUAUAAAGACACGACGAU | 20 |
| hsa-miR-424 | 3′ AAGUUUUGUACUUAACGACGAC | 21 |
| hsa-miR-497 | 3′ UGUUUGGUGUCACACGACGAC | 22 |

hsa = Homo sapiens

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing cells for application to a wound in a diabetic subject, the method comprising incubating the cells in the presence of an effective amount of a PKCδ inhibitor.

2. A method of treating a wound in a diabetic subject, the method comprising:
providing a cell derived from the subject;
incubating the cells in the presence of an effective amount of a PKCδ inhibitor; and
administering the cells to the wound.

3. The method of claim 2, wherein the cells are keratinocytes, fibroblasts, or a combination thereof.

4. The method of claim 2, wherein the cells are, or are derived from epithelial stem cells; human embryonic stem cells; induced pluripotent stem cells (iPS); bone-marrow-derived mesenchymal stem cells (BM-MSCs) or adipose-tissue-derived MSCs (ASCs).

5. The method of claim 2, wherein the cells are part of a split-thickness graft.

6. The method of claim 2, wherein the PKCδ inhibitor is selected from the group consisting of Rottlerin; PKC-412; and UCN-02; KAI-980, bisindolylmaleimide I, bisindolylmaleimide II, bisindolylmaleimide III, bisindolylmaleimide IV, calphostin C, chelerythrine chloride, ellagic Acid, Go 7874, Go 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, D-erythro-sphingosine, an inhibitory nucleic acid that specifically targets PKCδ, and an oligonucleotide mimic that mimics a PKCδ miRNA selected from the group consisting of miR-15a, 15b, 16, 195, 424, and 497.

7. (canceled)

8. (canceled)

9. The method of claim 7, wherein the inhibitory nucleic acid is 10 to 50 bases in length.

10. The method of claim 7, wherein the inhibitory nucleic acid comprises a base sequence at least 90% complementary to at least 10 bases of the PKCδ RNA sequence.

11. (canceled)

12. (canceled)

13. (canceled)

14. (canceled)

15. (canceled)

16. (canceled)

17. The method of claim 7, wherein the inhibitory nucleic acid or oligonucleotide mimic comprises one or more modifications comprising: a modified sugar moiety, a modified internucleoside linkage, a modified nucleotide and/or combinations thereof.

18. The method of claim 17, wherein the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof.

19. The method of claim 17, wherein the modified sugar moiety comprises a 2′-O-methoxyethyl modified sugar moiety, a 2′-methoxy modified sugar moiety, a 2′-O-alkyl modified sugar moiety, or a bicyclic sugar moiety.

20. The method of claim 17, wherein the inhibitory nucleic acid or oligonucleotide mimic comprises one or more of: 2′-OMe, 2′-F, LNA, PNA, FANA, ENA or morpholino modifications.

21. The method of claim 7, wherein the inhibitory nucleic acid is an antisense oligonucleotide, LNA molecule, PNA molecule, ribozyme or siRNA.

22. The method of claim 7, wherein the inhibitory nucleic acid is double stranded and comprises an overhang at one or both termini.

23. The method of claim 7, wherein the inhibitory nucleic acid is selected from the group consisting of antisense oligonucleotides and single- or double-stranded RNA interference (RNAi) compounds.

24. The method of claim 23, wherein the RNAi compound is selected from the group consisting of short interfering RNA (siRNA); or a short, hairpin RNA (shRNA); small RNA-induced gene activation (RNAa); and small activating RNAs (saRNAs).

25. (canceled)

26. The method of claim 1, wherein incubating the cells in the presence of an effective amount of a PKCδ inhibitor comprises expressing a dominant negative PKCδ (dnPKCδ) in the cells.

27. The method of claim 26, comprising transfecting the cells with a viral vector encoding the dnPKCδ.

28. The method of claim 27, wherein the viral vector is an adenoviral vector.

29. The method of claim 2, wherein the cells are administered in a carrier.

30. The method of claim 29, wherein the carrier is, or is applied to, a membrane.

31. The method of claim 29, wherein the carrier is liquid or semi-solid.

32. An isolated population of cells prepared by the method of claim 1.

33. The isolated population of cells of claim 32, for use in a method of treating a wound in a diabetic subject.

34. The isolated population of cells of claim 33, wherein the cells were obtained from the subject to be treated.
